SEQUENCE LISTING (187 sequences)

SEQ ID NO: 1 - DNA, 312 bp, Artificial Sequence
  synthetic
gacatccagatgacccagtctccttccaccctgtctgcatctgtaggagacagagtcaccatcacttgccgggccagtcagagtattagtaactggttggcctggtatcaacagaaaccagggaaagcccctaaactcctgatctataaggcgtctagtttagagagtggggtcccatcaaggttcagcggcagtggatctgggacagaattcactctcaccatcagcagcctgcagcctgatgattttgcaacttattactgccaacactattatggggctttcggccctgggaccaaagtggatatcaaa

SEQ ID NO: 2 - protein, 104 aa, Artificial Sequence
  synthetic
DIQMTQSPSTLSASVGDRVTITCRASQSISNWLAWYQQKPGKAPKLLIYKASSLESGVPSRFSGSGSGTEFTLTISSLQPDDFATYYCQHYYGAFGPGTKVDIK

SEQ ID NO: 3 - DNA, 18 bp, Artificial Sequence
  synthetic
cagagtattagtaactgg

SEQ ID NO: 4 - protein, 6 aa, Artificial Sequence
  synthetic
QSISNW

SEQ ID NO: 5 - DNA, 9 bp, Artificial Sequence
  synthetic
aaggcgtct

SEQ ID NO: 6 - protein, 3 aa, Artificial Sequence
  synthetic
KAS

SEQ ID NO: 7 - DNA, 18 bp, Artificial Sequence
  synthetic
caacactattatggggct

SEQ ID NO: 8 - protein, 6 aa, Artificial Sequence
  synthetic
QHYYGA

SEQ ID NO: 9 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccagcagagacctggccaggctcccaggctcctcatttatgatgcatccagcagggccactggcatcccagacaggttcagtggcggtgggtctgggacagacttcactctcaccatcagcagactggagcctgaagattttgcagtgtattactgtcagcagtatggtagctcaccattcactttcggccctgggaccaaagtggatatcaaa

SEQ ID NO: 10 - protein, 108 aa, Artificial Sequence
  synthetic
EIVLTQSPGTLSLSPGERATLSCRASQSVSSSYLAWYQQRPGQAPRLLIYDASSRATGIPDRFSGGGSGTDFTLTISRLEPEDFAVYYCQQYGSSPFTFGPGTKVDIK

SEQ ID NO: 11 - DNA, 21 bp, Artificial Sequence
  synthetic
cagagtgttagcagcagctac

SEQ ID NO: 12 - protein, 7 aa, Artificial Sequence
  synthetic
QSVSSSY

SEQ ID NO: 13 - DNA, 9 bp, Artificial Sequence
  synthetic
gatgcatcc

SEQ ID NO: 14 - protein, 3 aa, Artificial Sequence
  synthetic
DAS

SEQ ID NO: 15 - DNA, 27 bp, Artificial Sequence
  synthetic
cagcagtatggtagctcaccattcact

SEQ ID NO: 16 - protein, 9 aa, Artificial Sequence
  synthetic
QQYGSSPFT

SEQ ID NO: 17 - DNA, 330 bp, Artificial Sequence
  synthetic
gaaattgtgatgacgcagtctccaaccaccctgtctgtgtctccaggggaaagagccaccctctcctgcagggccagtcagagtgttagcagcaacttagcctggtaccaacagaaacctggccaggctctcagggtcctcatctatggtgcatccaccagggccactggtgtcccagccaggttcagaggcactgggtctgggacagacttcactctcaccatcactagtctgcagtctgaagattttgcagtttattactgtcagcagtataataagtggcccagagggaagaccaccttcggccaagggacacgactggagattaaa

SEQ ID NO: 18 - protein, 110 aa, Artificial Sequence
  synthetic
EIVMTQSPTTLSVSPGERATLSCRASQSVSSNLAWYQQKPGQALRVLIYGASTRATGVPARFRGTGSGTDFTLTITSLQSEDFAVYYCQQYNKWPRGKTTFGQGTRLEIK

SEQ ID NO: 19 - DNA, 18 bp, Artificial Sequence
  synthetic
cagagtgttagcagcaac

SEQ ID NO: 20 - protein, 6 aa, Artificial Sequence
  synthetic
QSVSSN

SEQ ID NO: 21 - DNA, 9 bp, Artificial Sequence
  synthetic
ggtgcatcc

SEQ ID NO: 22 - protein, 3 aa, Artificial Sequence
  synthetic
GAS

SEQ ID NO: 23 - DNA, 36 bp, Artificial Sequence
  synthetic
cagcagtataataagtggcccagagggaagaccacc

SEQ ID NO: 24 - protein, 12 aa, Artificial Sequence
  synthetic
QQYNKWPRGKTT

SEQ ID NO: 25 - DNA, 321 bp, Artificial Sequence
  synthetic
gacatccagatgacccagtctccatcctccctgtctgcatctgtaggagacagagtcaccatcacttgccgggcaagtcagaacattcgcagctacttaaattggtatcagcagaaaccagggaaagcccctaacctcctgattaatgttgcatccagtttgcaaagtggggtcccatcaaggttcagtggcagtggatctgggacagctttcactctcactatcagcaatctgcaacctgaagattttgtaatttattattgtcaacagagttacagtgacccattcactttcggccctgggaccaaagtggatatcaaa

SEQ ID NO: 26 - protein, 107 aa, Artificial Sequence
  synthetic
DIQMTQSPSSLSASVGDRVTITCRASQNIRSYLNWYQQKPGKAPNLLINVASSLQSGVPSRFSGSGSGTAFTLTISNLQPEDFVIYYCQQSYSDPFTFGPGTKVDIK

SEQ ID NO: 27 - DNA, 18 bp, Artificial Sequence
  synthetic
cagaacattcgcagctac

SEQ ID NO: 28 - protein, 6 aa, Artificial Sequence
  synthetic
QNIRSY

SEQ ID NO: 29 - DNA, 9 bp, Artificial Sequence
  synthetic
gttgcatcc

SEQ ID NO: 30 - protein, 3 aa, Artificial Sequence
  synthetic
VAS

SEQ ID NO: 31 - DNA, 27 bp, Artificial Sequence
  synthetic
caacagagttacagtgacccattcact

SEQ ID NO: 32 - protein, 9 aa, Artificial Sequence
  synthetic
QQSYSDPFT

SEQ ID NO: 33 - DNA, 312 bp, Artificial Sequence
  synthetic
gacatccagatgacccagtcgccttccaccctgtctgcatctgtaggagacagagtcaccatcacttgccgggccagtcagagtattagtaataggttggcctggtatcagcagaaaccagggaaagcccctaagttcctgatctataaggcgtctagtttagatagtggggtcccatcaaggttcagcggcagtggatctgggacagaattcactctcaccatcagcagcctgcaacctgatgattttgcaacttattactgccaacagtattatggggctttcggcggagggaccaaggtggagatcaaa

SEQ ID NO: 34 - protein, 104 aa, Artificial Sequence
  synthetic
DIQMTQSPSTLSASVGDRVTITCRASQSISNRLAWYQQKPGKAPKFLIYKASSLDSGVPSRFSGSGSGTEFTLTISSLQPDDFATYYCQQYYGAFGGGTKVEIK

SEQ ID NO: 35 - DNA, 18 bp, Artificial Sequence
  synthetic
cagagtattagtaatagg

SEQ ID NO: 36 - protein, 6 aa, Artificial Sequence
  synthetic
QSISNR

SEQ ID NO: 37 - DNA, 9 bp, Artificial Sequence
  synthetic
aaggcgtct

SEQ ID NO: 38 - protein, 3 aa, Artificial Sequence
  synthetic
KAS

SEQ ID NO: 39 - DNA, 18 bp, Artificial Sequence
  synthetic
caacagtattatggggct

SEQ ID NO: 40 - protein, 6 aa, Artificial Sequence
  synthetic
QQYYGA

SEQ ID NO: 41 - DNA, 324 bp, Artificial Sequence
  synthetic
gaaattgtgttgacgcagtctccaggcaccctgtctttgtctccaggggaaagagccaccctctcctgcagggccagtcagagtgttagcttcagctacttagcctggtaccagcagaaacctggccaggctcccaggctcctcatctatggtgcatccagcagggccgctggcatcccagacaggttcagtggcagtgggtctgggacagacttcactctcaccatcagcagactggagcctgaagattttgcagtgtattactgtcagcagtatggtagttcaccgtacacttttggccaggggaccaagctggagatcaaa

SEQ ID NO: 42 - protein, 108 aa, Artificial Sequence
  synthetic
EIVLTQSPGTLSLSPGERATLSCRASQSVSFSYLAWYQQKPGQAPRLLIYGASSRAAGIPDRFSGSGSGTDFTLTISRLEPEDFAVYYCQQYGSSPYTFGQGTKLEIK

SEQ ID NO: 43 - DNA, 21 bp, Artificial Sequence
  synthetic
cagagtgttagcttcagctac

SEQ ID NO: 44 - protein, 7 aa, Artificial Sequence
  synthetic
QSVSFSY

SEQ ID NO: 45 - DNA, 9 bp, Artificial Sequence
  synthetic
ggtgcatcc

SEQ ID NO: 46 - protein, 3 aa, Artificial Sequence
  synthetic
GAS

SEQ ID NO: 47 - DNA, 27 bp, Artificial Sequence
  synthetic
cagcagtatggtagttcaccgtacact

SEQ ID NO: 48 - protein, 9 aa, Artificial Sequence
  synthetic
QQYGSSPYT

SEQ ID NO: 49 - DNA, 312 bp, Artificial Sequence
  synthetic
gacatccagatgacccagtctccttccaccctgtctgcatctgtaggagacagagtcaccatcacttgccgggccagtcagagtattggtaactggttggcctggtatcagcagaaaccagggaaagcccctaaactcctgatctatcaggcgtctagtttagacaggggggtcccatcaaggttcagcggaagtggatcagggacagaattcactctcaccatcagcagcctgcagcctgatgattatgcaacttattactgccaacattattatggggctttcggcggagggaccaaggtggagatcaaa

SEQ ID NO: 50 - protein, 104 aa, Artificial Sequence
  synthetic
DIQMTQSPSTLSASVGDRVTITCRASQSIGNWLAWYQQKPGKAPKLLIYQASSLDRGVPSRFSGSGSGTEFTLTISSLQPDDYATYYCQHYYGAFGGGTKVEIK

SEQ ID NO: 51 - DNA, 18 bp, Artificial Sequence
  synthetic
cagagtattggtaactgg

SEQ ID NO: 52 - protein, 6 aa, Artificial Sequence
  synthetic
QSIGNW

SEQ ID NO: 53 - DNA, 9 bp, Artificial Sequence
  synthetic
caggcgtct

SEQ ID NO: 54 - protein, 3 aa, Artificial Sequence
  synthetic
QAS

SEQ ID NO: 55 - DNA, 18 bp, Artificial Sequence
  synthetic
caacattattatggggct

SEQ ID NO: 56 - protein, 6 aa, Artificial Sequence
  synthetic
QHYYGA

SEQ ID NO: 57 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccggcagaaacctggccaggctcccaggctcctcatctatggtgcatccagcagggccactggcatcccagacaggttcagtggcagtgggtctgggacagacttcactctcatcatcagcagactggagcctgaagattttgcagtgtattactgtcagcagtatggaagttcaccattcactttcggccctgggaccaaagtggatatcaaa

SEQ ID NO: 58 - protein, 108 aa, Artificial Sequence
  synthetic
EIVLTQSPGTLSLSPGERATLSCRASQSVSSSYLAWYRQKPGQAPRLLIYGASSRATGIPDRFSGSGSGTDFTLIISRLEPEDFAVYYCQQYGSSPFTFGPGTKVDIK

SEQ ID NO: 59 - DNA, 21 bp, Artificial Sequence
  synthetic
cagagtgttagcagcagctac

SEQ ID NO: 60 - protein, 7 aa, Artificial Sequence
  synthetic
QSVSSSY

SEQ ID NO: 61 - DNA, 9 bp, Artificial Sequence
  synthetic
ggtgcatcc

SEQ ID NO: 62 - protein, 3 aa, Artificial Sequence
  synthetic
GAS

SEQ ID NO: 63 - DNA, 27 bp, Artificial Sequence
  synthetic
cagcagtatggaagttcaccattcact

SEQ ID NO: 64 - protein, 9 aa, Artificial Sequence
  synthetic
QQYGSSPFT

SEQ ID NO: 65 - DNA, 312 bp, Artificial Sequence
  synthetic
gacatccagatgacccagtctccctccgccctgtctgcatctgtaggagacagagtcaccatcacttgccgggccagtcagagtatcagtagctggttggcctggtatcagcagaagccagggaaagcccctaagctcctgatctatcaggcgtctactttagacagaggggtcccatcaaggttcagcggcagtggatctgggacagaattcactctcaccatcagcagcctgcagcctgatgattttgcaacttattactgccaacagtattatggggccttcggccaagggacacgactggagattaaa

SEQ ID NO: 66 - protein, 104 aa, Artificial Sequence
  synthetic
DIQMTQSPSALSASVGDRVTITCRASQSISSWLAWYQQKPGKAPKLLIYQASTLDRGVPSRFSGSGSGTEFTLTISSLQPDDFATYYCQQYYGAFGQGTRLEIK

SEQ ID NO: 67 - DNA, 18 bp, Artificial Sequence
  synthetic
cagagtatcagtagctgg

SEQ ID NO: 68 - protein, 6 aa, Artificial Sequence
  synthetic
QSISSW

SEQ ID NO: 69 - DNA, 9 bp, Artificial Sequence
  synthetic
caggcgtct

SEQ ID NO: 70 - protein, 3 aa, Artificial Sequence
  synthetic
QAS

SEQ ID NO: 71 - DNA, 18 bp, Artificial Sequence
  synthetic
caacagtattatggggcc

SEQ ID NO: 72 - protein, 6 aa, Artificial Sequence
  synthetic
QQYYGA

SEQ ID NO: 73 - DNA, 324 bp, Artificial Sequence
  synthetic
gaaattgtgttgacgcagtctccaggcaccctgtctttgtctccaggggaaagagccaccctctcctgcagggccagtcaaagtgttagcaggaactacttagcctggtatcaacagaaacctggccaggctcccaggctcctcatctatggtgcatccagcagggccagtggcatcccggacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 74 - protein, 108 aa, Artificial Sequence
  synthetic
EIVLTQSPGTLSLSPGERATLSCRASQSVSRNYLAWYQQKPGQAPRLLIYGASSRASGIPDRFSGSGSGTDFTLTISRLEPEDFAVYYCQQYGSSPYTFGQGTKLEIK

SEQ ID NO: 75 - DNA, 21 bp, Artificial Sequence
  synthetic
caaagtgttagcaggaactac

SEQ ID NO: 76 - protein, 7 aa, Artificial Sequence
  synthetic
QSVSRNY

SEQ ID NO: 77 - DNA, 9 bp, Artificial Sequence
  synthetic
ggtgcatcc

SEQ ID NO: 78 - protein, 3 aa, Artificial Sequence
  synthetic
GAS

SEQ ID NO: 79 - DNA, 27 bp, Artificial Sequence
  synthetic
cagcagtatggtagctcaccgtacact

SEQ ID NO: 80 - protein, 9 aa, Artificial Sequence
  synthetic
QQYGSSPYT

SEQ ID NO: 81 - DNA, 312 bp, Artificial Sequence
  synthetic
gacatccagatgacccagtctccttccaccctgtctgcatctgtaggagacagagtcaccatcacttgccgggccagtcagagtattagtaactggttggcctggtatcagcagaaaccagggaaagcccctaagctcctgatctataaggcgtctagtttagacagtggggtcccatcaaggttcagcggcagtggatctgggacagaattcactctcaccatcagcagcctgcagcctgatgattttgcaacttattactgccaacagttttatggcgctttcggccctgggaccaaagtggatatcaaa

SEQ ID NO: 82 - protein, 104 aa, Artificial Sequence
  synthetic
DIQMTQSPSTLSASVGDRVTITCRASQSISNWLAWYQQKPGKAPKLLIYKASSLDSGVPSRFSGSGSGTEFTLTISSLQPDDFATYYCQQFYGAFGPGTKVDIK

SEQ ID NO: 83 - DNA, 18 bp, Artificial Sequence
  synthetic
cagagtattagtaactgg

SEQ ID NO: 84 - protein, 6 aa, Artificial Sequence
  synthetic
QSISNW

SEQ ID NO: 85 - DNA, 9 bp, Artificial Sequence
  synthetic
aaggcgtct

SEQ ID NO: 86 - protein, 3 aa, Artificial Sequence
  synthetic
KAS

SEQ ID NO: 87 - DNA, 18 bp, Artificial Sequence
  synthetic
caacagttttatggcgct

SEQ ID NO: 88 - protein, 6 aa, Artificial Sequence
  synthetic
QQFYGA

SEQ ID NO: 89 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtacacttttggccaggggaccaagctggagatcaaa

SEQ ID NO: 90 - protein, 108 aa, Artificial Sequence
  synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 91 - DNA, 21 bp, Artificial Sequence
  synthetic
cagagtgttagcagcagctac

SEQ ID NO: 92 - protein, 7 aa, Artificial Sequence
  synthetic
QSVSSSY

SEQ ID NO: 93 - DNA, 9 bp, Artificial Sequence
  synthetic
ggtgcatcc

SEQ ID NO: 94 - protein, 3 aa, Artificial Sequence
  synthetic
GAS

SEQ ID NO: 95 - DNA, 27 bp, Artificial Sequence
  synthetic
cagcagtatggtagctcaccgtacact

SEQ ID NO: 96 - protein, 9 aa, Artificial Sequence
  synthetic
QQYGSSPYT

SEQ ID NO: 97 - DNA, 339 bp, Artificial Sequence
  synthetic
gacatcgtgatgacccagtctccagactccctggctgtgtctctgggcgagagggccaccatcaactgcaagtccagtcagagtgttttatacagctccaataataaaaactacttagcttggttccagcagaaaccaggacagcctcctaagcttctcatttactgggcatctctccgggaatccggggtctctgaccgattcagtggcagcgggtctgggacagatttcactctcaccatcagcggcctgcaggctgaagatgtggccgtttattactgtcagcaatattatagtattctgtggacgttcggccaagggaccaaggtggaaatcaaa

SEQ ID NO: 98 - protein, 113 aa, Artificial Sequence
  synthetic
DIVMTQSPDSLAVSLGERATINCKSSQSVLYSSNNKNYLAWFQQKPGQPPKLLIYWASLRESGVSDRFSGSGSGTDFTLTISGLQAEDVAVYYCQQYYSILWTFGQGTKVEIK

SEQ ID NO: 99 - DNA, 36 bp, Artificial Sequence
  synthetic
cagagtgttttatacagctccaataataaaaactac

SEQ ID NO: 100 - protein, 12 aa, Artificial Sequence
  synthetic
QSVLYSSNNKNY

SEQ ID NO: 101 - DNA, 9 bp, Artificial Sequence
  synthetic
tgggcatct

SEQ ID NO: 102 - protein, 3 aa, Artificial Sequence
  synthetic
WAS

SEQ ID NO: 103 - DNA, 27 bp, Artificial Sequence
  synthetic
cagcaatattatagtattctgtggacg

SEQ ID NO: 104 - protein, 9 aa, Artificial Sequence
  synthetic
QQYYSILWT

SEQ ID NO: 105 - DNA, 324 bp, Artificial Sequence
  synthetic
gaaattgtgttgacgcagtctccaggcaccctgtctttgtctccaggggaaagagccaccctctcctgtagggccagtcagagtgttggcaacaactacttagcctggttccagcagaaacctggccaggctcccaggctcctcatctatggtgcatccagtagggccactggcatcccagacaggttcagtggcagtgggtctgggacagacttcactctcaccatcagcagactggagcctgaagattttgtagtgtattactgtcaacagtatagtaggtcaccgtatatttttggccaggggaccaagctggagatcaaa

SEQ ID NO: 106 - protein, 108 aa, Artificial Sequence
  synthetic
EIVLTQSPGTLSLSPGERATLSCRASQSVGNNYLAWFQQKPGQAPRLLIYGASSRATGIPDRFSGSGSGTDFTLTISRLEPEDFVVYYCQQYSRSPYIFGQGTKLEIK

SEQ ID NO: 107 - DNA, 21 bp, Artificial Sequence
  synthetic
cagagtgttggcaacaactac

SEQ ID NO: 108 - protein, 7 aa, Artificial Sequence
  synthetic
QSVGNNY

SEQ ID NO: 109 - DNA, 9 bp, Artificial Sequence
  synthetic
ggtgcatcc

SEQ ID NO: 110 - protein, 3 aa, Artificial Sequence
  synthetic
GAS

SEQ ID NO: 111 - DNA, 27 bp, Artificial Sequence
  synthetic
caacagtatagtaggtcaccgtatatt

SEQ ID NO: 112 - protein, 9 aa, Artificial Sequence
  synthetic
QQYSRSPYI

SEQ ID NO: 113 - DNA, 312 bp, Artificial Sequence
  synthetic
gacatccagatgacccagtctccttccaccctgtctgcatctgtaggagacagagtcaccatcacttgccgggccagtcagaatattaatagctggttggcctggtatcaacagaaaccagggaaagcccctaagttcctgatctataaggcgtctagtttagaaagtggggtcccatcaaggttcagcggcagtggatctgggacagaattcactctcaccatcagcagcctgcaacctgatgattttgcaacttattactgccaacagtattggggcactttcggcggagggaccaaggtggagatcaaa

SEQ ID NO: 114 - protein, 104 aa, Artificial Sequence
  synthetic
DIQMTQSPSTLSASVGDRVTITCRASQNINSWLAWYQQKPGKAPKFLIYKASSLESGVPSRFSGSGSGTEFTLTISSLQPDDFATYYCQQYWGTFGGGTKVEIK

SEQ ID NO: 115 - DNA, 18 bp, Artificial Sequence
  synthetic
cagaatattaatagctgg

SEQ ID NO: 116 - protein, 6 aa, Artificial Sequence
  synthetic
QNINSW

SEQ ID NO: 117 - DNA, 9 bp, Artificial Sequence
  synthetic
aaggcgtct

SEQ ID NO: 118 - protein, 3 aa, Artificial Sequence
  synthetic
KAS

SEQ ID NO: 119 - DNA, 18 bp, Artificial Sequence
  synthetic
caacagtattggggcact

SEQ ID NO: 120 - protein, 6 aa, Artificial Sequence
  synthetic
QQYWGT

SEQ ID NO: 121 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcgtggccactggcatcccagacaggttcagtgccagtgggtctgggacagacttcactctcaccatcagcagactggagcctgaagattttgcagtgtattactgccagcagtatggtagctcacctcggacgttcggccaagggaccaaggtggaaatcaaa

SEQ ID NO: 122 - protein, 108 aa, Artificial Sequence
  synthetic
EIVLTQSPGTLSLSPGERATLSCRASQSVSSSYLAWYQQKPGQAPRLLIYGASSVATGIPDRFSASGSGTDFTLTISRLEPEDFAVYYCQQYGSSPRTFGQGTKVEIK

SEQ ID NO: 123 - DNA, 21 bp, Artificial Sequence
  synthetic
cagagtgttagcagcagctac

SEQ ID NO: 124 - protein, 7 aa, Artificial Sequence
  synthetic
QSVSSSY

SEQ ID NO: 125 - DNA, 9 bp, Artificial Sequence
  synthetic
ggtgcatcc

SEQ ID NO: 126 - protein, 3 aa, Artificial Sequence
  synthetic
GAS

SEQ ID NO: 127 - DNA, 27 bp, Artificial Sequence
  synthetic
cagcagtatggtagctcacctcggacg

SEQ ID NO: 128 - protein, 9 aa, Artificial Sequence
  synthetic
QQYGSSPRT

SEQ ID NO: 129 - DNA, 312 bp, Artificial Sequence
  synthetic
gacatccagatgacccagtcaccttccaccctgtctgcatctgtaggagacagagtcaccatcacttgtcgggccagtcagagtattagtagccggctggcctggtatcagcagaaaccagggaaagcccctaagttccttatctatcaggcgtctagtttagagagaggggtcccatcgaggttcagcggcagtggctctgggacagaattcactctcaccatcagcagcctgcagcctgatgattttgcaacttattactgccaacactattggggcaccttcggcccagggacacgactggagattaaa

SEQ ID NO: 130 - protein, 104 aa, Artificial Sequence
  synthetic
DIQMTQSPSTLSASVGDRVTITCRASQSISSRLAWYQQKPGKAPKFLIYQASSLERGVPSRFSGSGSGTEFTLTISSLQPDDFATYYCQHYWGTFGPGTRLEIK

SEQ ID NO: 131 - DNA, 18 bp, Artificial Sequence
  synthetic
cagagtattagtagccgg

SEQ ID NO: 132 - protein, 6 aa, Artificial Sequence
  synthetic
QSISSR

SEQ ID NO: 133 - DNA, 9 bp, Artificial Sequence
  synthetic
caggcgtct

SEQ ID NO: 134 - protein, 3 aa, Artificial Sequence
  synthetic
QAS

SEQ ID NO: 135 - DNA, 18 bp, Artificial Sequence
  synthetic
caacactattggggcacc

SEQ ID NO: 136 - protein, 6 aa, Artificial Sequence
  synthetic
QHYWGT

SEQ ID NO: 137 - DNA, 324 bp, Artificial Sequence
  synthetic
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccccggacgttcggccaagggaccaaggtggaaatcaaa

SEQ ID NO: 138 - protein, 108 aa, Artificial Sequence
  synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 139 - DNA, 21 bp, Artificial Sequence
  synthetic
cagagtgttagcagcagctac

SEQ ID NO: 140 - protein, 7 aa, Artificial Sequence
  synthetic
QSVSSSY

SEQ ID NO: 141 - DNA, 9 bp, Artificial Sequence
  synthetic
ggtgcatcc

SEQ ID NO: 142 - protein, 3 aa, Artificial Sequence
  synthetic
GAS

SEQ ID NO: 143 - DNA, 27 bp, Artificial Sequence
  synthetic
cagcagtatggtagctcaccccggacg

SEQ ID NO: 144 - protein, 9 aa, Artificial Sequence
  synthetic
QQYGSSPRT

SEQ ID NO: 145 - DNA, 345 bp, Artificial Sequence
  synthetic
gacatcgtgatgacccagtctccagactccctggctgtgtctctgggcgcgagggccaccatcaactgcaagtccagtcagagtgttttatacagctccaacaataagaactacttagcttggttccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcaggaatattttactactcctccggggtggacgttcggccaagggaccaaggtggaaatcaaa

SEQ ID NO: 146 - protein, 115 aa, Artificial Sequence
  synthetic
DIVMTQSPDSLAVSLGARATINCKSSQSVLYSSNNKNYLAWFQQKPGQPPKLLIYWASTRESGVPDRFSGSGSGTDFTLTISSLQAEDVAVYYCQEYFTTPPGWTFGQGTKVEIK

SEQ ID NO: 147 - DNA, 36 bp, Artificial Sequence
  synthetic
cagagtgttttatacagctccaacaataagaactac

SEQ ID NO: 148 - protein, 12 aa, Artificial Sequence
  synthetic
QSVLYSSNNKNY

SEQ ID NO: 149 - DNA, 9 bp, Artificial Sequence
  synthetic
tgggcatct

SEQ ID NO: 150 - protein, 3 aa, Artificial Sequence
  synthetic
WAS

SEQ ID NO: 151 - DNA, 33 bp, Artificial Sequence
  synthetic
caggaatattttactactcctccggggtggacg

SEQ ID NO: 152 - protein, 11 aa, Artificial Sequence
  synthetic
QEYFTTPPGWT

SEQ ID NO: 153 - DNA, 324 bp, Artificial Sequence
  synthetic
gaaattgtgttgacgcagtctccaggcaccctgtctttgtctccaggggaaagagccaccctctcctgcagggccagtcagagtgttagcagcaactacttagcctggttccagcagagacctggccaggctcccaggctcctcatctatggtgcatcccgcagggccactggcatcccagacaggttccgtggcagtggggctgggacagacttcactctcaccatcagcagactggagcctgaagattttgcagtgtattactgtcagcagtatattaggtcaccgtatatttttggccaggggaccaagctggagatcaaa

SEQ ID NO: 154 - protein, 108 aa, Artificial Sequence
  synthetic
EIVLTQSPGTLSLSPGERATLSCRASQSVSSNYLAWFQQRPGQAPRLLIYGASRRATGIPDRFRGSGAGTDFTLTISRLEPEDFAVYYCQQYIRSPYIFGQGTKLEIK

SEQ ID NO: 155 - DNA, 21 bp, Artificial Sequence
  synthetic
cagagtgttagcagcaactac

SEQ ID NO: 156 - protein, 7 aa, Artificial Sequence
  synthetic
QSVSSNY

SEQ ID NO: 157 - DNA, 9 bp, Artificial Sequence
  synthetic
ggtgcatcc

SEQ ID NO: 158 - protein, 3 aa, Artificial Sequence
  synthetic
GAS

SEQ ID NO: 159 - DNA, 27 bp, Artificial Sequence
  synthetic
cagcagtatattaggtcaccgtatatt

SEQ ID NO: 160 - protein, 9 aa, Artificial Sequence
  synthetic
QQYIRSPYI

SEQ ID NO: 161 - DNA, 345 bp, Artificial Sequence
  synthetic
gacatcgtgatgacccagtctccggactccctggctgtgtctctgggcgagagggccaccatcaactgcaagtccagccagagtattttatacagctccaacaataagaactacttagcttggttccagcagaaaccaggacagcctcctaggctgctcatttactgggcatctacccgggaatccggggtccctgaccgattcagtggcagcgggtctgggacagatttcactctcaccatcagcagcctgcaggctgaagatgtggcagtttattactgtcaggaatattatgttactcctcccgggtggactttcggccctgggaccaaagtggatatcaaa

SEQ ID NO: 162 - protein, 115 aa, Artificial Sequence
  synthetic
DIVMTQSPDSLAVSLGERATINCKSSQSILYSSNNKNYLAWFQQKPGQPPRLLIYWASTRESGVPDRFSGSGSGTDFTLTISSLQAEDVAVYYCQEYYVTPPGWTFGPGTKVDIK

SEQ ID NO: 163 - DNA, 36 bp, Artificial Sequence
  synthetic
cagagtattttatacagctccaacaataagaactac

SEQ ID NO: 164 - protein, 12 aa, Artificial Sequence
  synthetic
QSILYSSNNKNY

SEQ ID NO: 165 - DNA, 9 bp, Artificial Sequence
  synthetic
tgggcatct

SEQ ID NO: 166 - protein, 3 aa, Artificial Sequence
  synthetic
WAS

SEQ ID NO: 167 - DNA, 33 bp, Artificial Sequence
  synthetic
caggaatattatgttactcctcccgggtggact

SEQ ID NO: 168 - protein, 11 aa, Artificial Sequence
  synthetic
QEYYVTPPGWT

SEQ ID NO: 169 - DNA, 324 bp, Artificial Sequence
  synthetic
gaaattgtgttgacgcagtctccaggcaccctgtctttgtctccaggggaaagagccaccctctcctgcagggccagtcagagtgttagcagcaactacttagcctggttccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatcgtagctcaccgtacatttttggccaggggaccaagctggagatcaaa

SEQ ID NO: 170 - protein, 108 aa, Artificial Sequence
  synthetic
EIVLTQSPGTLSLSPGERATLSCRASQSVSSNYLAWFQQKPGQAPRLLIYGASSRATGIPDRFSGSGSGTDFTLTISRLEPEDFAVYYCQQYRSSPYIFGQGTKLEIK

SEQ ID NO: 171 - DNA, 21 bp, Artificial Sequence
  synthetic
cagagtgttagcagcaactac

SEQ ID NO: 172 - protein, 7 aa, Artificial Sequence
  synthetic
QSVSSNY

SEQ ID NO: 173 - DNA, 9 bp, Artificial Sequence
  synthetic
ggtgcatcc

SEQ ID NO: 174 - protein, 3 aa, Artificial Sequence
  synthetic
GAS

SEQ ID NO: 175 - DNA, 27 bp, Artificial Sequence
  synthetic
cagcaatatcgtagctcaccgtacatt

SEQ ID NO: 176 - protein, 9 aa, Artificial Sequence
  synthetic
QQYRSSPYI

SEQ ID NO: 177 - protein, 12 aa, Artificial Sequence
  synthetic
  VARIANT 1, 2, 3, 4, 5, 6, 7, 8, 9, 10, 11, 12: Xaa = Any Amino Acid
XXXXXXXXXXXX

SEQ ID NO: 178 - protein, 3 aa, Artificial Sequence
  synthetic
  VARIANT 1, 2, 3: Xaa = Any Amino Acid
XXX

SEQ ID NO: 179 - protein, 12 aa, Artificial Sequence
  synthetic
  VARIANT 1, 2, 3, 4, 5, 6, 7, 8, 9, 10, 11, 12: Xaa = Any Amino Acid
XXXXXXXXXXXX

SEQ ID NO: 180 - protein, 7 aa, Artificial Sequence
  synthetic
  VARIANT 1, 2, 3, 4, 5, 6, 7: Xaa = Any Amino Acid
XXXXXXX

SEQ ID NO: 181 - protein, 3 aa, Artificial Sequence
  synthetic
  VARIANT 1, 2, 3: Xaa = Any Amino Acid
XXX

SEQ ID NO: 182 - protein, 9 aa, Artificial Sequence
  synthetic
  VARIANT 1, 2, 3, 4, 5, 6, 7, 8, 9: Xaa = Any Amino Acid
XXXXXXXXX

SEQ ID NO: 183 - protein, 327 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 184 - protein, 326 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 185 - protein, 326 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 186 - protein, 326 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 187 - protein, 326 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

(19) United States
(12) Patent Application Publication
(10) Pub. No.: US 2019/0002555 A1
(43) Pub. Date: Jan. 3, 2019

(54) ANTIGEN-BINDING PROTEINS TO MARINOBUFAGENIN

(71) Applicant: REGENERON PHARMACEUTICALS, INC., Tarrytown, NY (US)

(72) Inventors: Michael E. DUNN, Midland Park, NJ (US); Lori C. MORTON, Chappaqua, NY (US); Yonaton RAY, Bergenfield, NJ (US); Jee H. KIM, Ardsley, NY (US)

(73) Assignee: REGENERON PHARMACEUTICALS, INC., Tarrytown, NY (US)

(21) Appl. No.: 15/746,565

(22) PCT Filed: Jul. 21, 2016
(86) PCT No.: PCT/US2016/043360

§ 371 Date: Jan. 22, 2018

Related U.S. Application Data
(60) Provisional application No. 62/195,889, filed on Jul. 23, 2015.

(51) Int. Cl. C07K 16/26; A61K 39/395; A61K 45/06

(52) U.S. Cl. CPC C07K 16/26; A61K 39/3955; A61K 45/06; A61K 2039/505; C07K 2317/76; C07K 2317/92; C07K 2317/94; C07K 2317/21

(57) ABSTRACT

The present invention provides monoclonal antigen-binding proteins that bind to the cardiac glycoside marinobufagenin (MBG), and methods of use. In various embodiments of the invention, the antigen-binding proteins are fully human antigen-binding proteins that bind to MBG. In some embodiments, the antigen-binding proteins of the invention are useful for inhibiting or neutralizing MBG activity, thus providing a means of treating or preventing a MBG-associated disease or disorder selected from the group consisting of hypertension, myocardial fibrosis, uremic cardiomyopathy, heart failure, myocardial infarction, renal failure, renal fibrosis and pre-eclampsia.

### FIELD OF THE INVENTION

The present invention is related to human antigen-binding proteins and antigen-binding fragments thereof that specifically bind to the cardiac glycoside marinobufagenin (MBG), and therapeutic and diagnostic methods of using those antigen-binding proteins.

### STATEMENT OF RELATED ART

Marinobufagenin (MBG) is an endogenous cardiac glycoside produced by the adrenal gland and belongs to a group of hormones that can bind and inhibit Na+/K+ ATPase (Fedorova et al 2002; Circulation 105: 1122-1127). Na+/K+ ATPase is a ubiquitously expressed pump that actively transports sodium and potassium ions across the plasma membrane to keep a high concentration of intracellular K⁺ ions and a low concentration of intracellular Na⁺ ions and maintains the electrical membrane potential in response to ionic flux. MBG, through inhibition of Na+/K+ ATPase, can regulate sodium levels, contributing to sodium imbalance in the blood and the renal system.

MBG is implicated in volume expansion hypertension, pre-eclampsia, heart failure, uremic cardiomyopathy and diabetes. Circulating MBG levels are found to be elevated in urine and serum in humans with cardiovascular disease (Uddin et al 2012, Transl. Res. 160: 99-113; Fedorova et al 2015, J. Hypertens. 33: 534-541). In rodents, chronic administration of MBG was found to cause hypertension, cardiac fibrosis, renal fibrosis, and altered glucose disposal (Fedorova et al 2002, Circulation 105: 1122-1127; Vu et al 2005, Am. J. Nephrol. 25: 520-528; Yoshika et al 2007, Hypertension 49: 209-214). Inhibition of MBG by an anti-MBG antibody was found to improve outcome in rodent models of hypertension, uremic cardiomyopathy and fibrosis (Fedorova et al 2005, J. Hypertens. 23: 835-842; Fedorova et al 2008, J. Hypertens. 26: 2414-2425; Haller et al 2012, Am. J. Hypertens. 25: 690-696).

U.S. Pat. No. 8,038,997 describes hybridoma cell lines and monoclonal antibodies produced by hybridomas that specifically bind to MBG and methods to diagnose and reduce blood pressure.

Fully human antigen-binding proteins, including fully human antigen-binding proteins that specifically bind to MBG with high affinity and inhibit its activity have not been described in prior art and could be important in the prevention and treatment of cardiovascular disease.

### BRIEF SUMMARY OF THE INVENTION

The present invention provides antigen-binding proteins that bind marinobufagenin (MBG). The antigen-binding proteins of the present invention are useful, inter alia, for inhibiting or neutralizing the activity of MBG. In some embodiments, the antigen-binding proteins are useful for blocking binding of MBG to Na+/K+ ATPase. In some embodiments, the antigen-binding proteins function by inhibiting MBG activity and reducing blood pressure. In certain embodiments, the antigen-binding proteins are useful in preventing, treating or ameliorating at least one symptom of a MBG-associated disease or disorder (e.g., cardiovascular disease) in a subject. In certain embodiments, the antigen-binding proteins may be administered prophylactically or therapeutically to a subject having or at risk of having cardiovascular disease (e.g., hypertension, cardiomyopathy or pre-eclampsia).

The antigen-binding proteins of the invention may comprise an antigen-binding domain and a Fc domain (for example, of an IgG1 or IgG4 antibody) or may comprise only an antigen-binding portion (for example, a Fab, F(ab)₂ or scFv fragment), and may be modified to affect functionality, e.g., to increase persistence in the host or to eliminate residual effector functions (Reddy et al., 2000, J. Immunol. 164:1925-1933). In certain embodiments, the antigen-binding proteins may be bispecific.

In a first aspect, the present invention provides isolated recombinant antigen-binding proteins that bind specifically to MBG. In some embodiments, the antigen-binding proteins are fully human antigen-binding proteins or monoclonal antibodies. In certain embodiments, the present invention provides antigen-binding proteins comprising an antigen-binding domain and a Fc domain. In certain embodiments, the antigen-binding domain comprises an immunoglobulin variable region comprising complementarity determining regions (CDRs) as described herein. In certain embodiments, the immunoglobulin variable region is a light chain variable region comprising three light chain CDRs as described herein.

In certain embodiments, the present invention provides antigen-binding proteins that specifically bind to MBG, wherein the antigen-binding protein comprises a first variable region (VR1) and a second variable region (VR2), wherein VR1 comprises three CDRs (CDR1, CDR2 and CDR3), and VR2 comprises three CDRs (CDR4, CDR5 and CDR6) as described herein. In certain embodiments, VR1 is a heavy chain variable region and VR2 is a light chain variable region. In certain embodiments, VR1 is a light chain variable region and VR2 is a light chain variable region.

Exemplary anti-MBG antigen-binding proteins of the present invention are listed in Tables 1 and 2 herein. Table 1 sets forth the amino acid sequence identifiers of the first and second variable regions (VRs) (VR1 and VR2), and CDRs (CDR1, CDR2, CDR3, CDR4, CDR5 and CDR6) of exemplary anti-MBG antigen-binding proteins. Table 2 sets forth the nucleic acid sequence identifiers of the VR1, VR2, CDR1, CDR2 CDR3, CDR4, CDR5 and CDR6 of exemplary anti-MBG antigen-binding proteins.

The present invention provides antigen-binding proteins, comprising a VR1 comprising an amino acid sequence selected from any of the VR1 amino acid sequences listed in Table 1, or a substantially similar sequence thereof having at least 90%, at least 95%, at least 98% or at least 99% sequence identity thereto.

The present invention also provides antigen-binding proteins, comprising a VR2 comprising an amino acid sequence selected from any of the VR2 amino acid sequences listed in Table 1, or a substantially similar sequence thereof having at least 90%, at least 95%, at least 98% or at least 99% sequence identity thereto.

The present invention also provides antigen-binding proteins, comprising a VR1 and a VR2 amino acid sequence pair (VR1/VR2) comprising any of the VR1 amino acid sequences listed in Table 1 paired with any of the VR2 amino acid sequences listed in Table 1. According to certain embodiments, the present invention provides antigen-binding proteins, comprising a VR1/VR2 amino acid sequence pair contained within any of the exemplary anti-MBG antigen-binding proteins listed in Table 1. In certain embodiments, the VR1/VR2 amino acid sequence pair is selected from the group consisting of SEQ ID NOs: 2/10, 18/26, 34/42, 50/58, 66/74, 82/90, 98/106, 114/122, 130/138, 146/154, and 162/170. In certain embodiments, the VR1/VR2 amino acid sequence pair is selected from one of SEQ ID NOs: 2/10 (e.g., H4H14357P), 50/58 (e.g., H4H14371P), or 98/106 (e.g., H4H14401P).

The present invention also provides antigen-binding proteins, comprising a CDR1 comprising an amino acid sequence selected from any of the CDR1 amino acid sequences listed in Table 1 or a substantially similar sequence thereof having at least 90%, at least 95%, at least 98% or at least 99% sequence identity.

The present invention also provides antigen-binding proteins, comprising a CDR2 comprising an amino acid sequence selected from any of the CDR2 amino acid sequences listed in Table 1 or a substantially similar sequence thereof having at least 90%, at least 95%, at least 98% or at least 99% sequence identity.

The present invention also provides antigen-binding proteins, comprising a CDR3 comprising an amino acid sequence selected from any of the CDR3 amino acid sequences listed in Table 1 or a substantially similar sequence thereof having at least 90%, at least 95%, at least 98% or at least 99% sequence identity.

The present invention also provides antigen-binding proteins, comprising a CDR4 comprising an amino acid sequence selected from any of the CDR4 amino acid sequences listed in Table 1 or a substantially similar sequence thereof having at least 90%, at least 95%, at least 98% or at least 99% sequence identity.

The present invention also provides antigen-binding proteins, comprising a CDR5 comprising an amino acid sequence selected from any of the CDR5 amino acid sequences listed in Table 1 or a substantially similar sequence thereof having at least 90%, at least 95%, at least 98% or at least 99% sequence identity.

The present invention also provides antigen-binding proteins, comprising a CDR6 comprising an amino acid sequence selected from any of the CDR6 amino acid sequences listed in Table 1 or a substantially similar sequence thereof having at least 90%, at least 95%, at least 98% or at least 99% sequence identity.

In certain embodiments, the antigen-binding domain comprises an immunoglobulin variable region comprising three CDRs (CDR1, CDR2 and CDR3), wherein:

(a) CDR1 comprises:
(i) an amino acid sequence selected from the group consisting of SEQ ID NOs: 4, 20, 36, 52, 68, 84, 100, 116, 132, 148, and 164;
(ii) an amino acid sequence with 90% identity to a sequence selected from the group consisting of SEQ ID NOs: 4, 20, 36, 52, 68, 84, 100, 116, 132, 148, and 164; or
(iii) an amino acid sequence with 3, 2 or 1 amino acid difference to a sequence selected from the group consisting of SEQ ID NOs: 4, 20, 36, 52, 68, 84, 100, 116, 132, 148, and 164;

(b) CDR2 comprises:
(iv) an amino acid sequence selected from the group consisting of SEQ ID NOs: 6, 22, 38, 54, 70, 86, 102, 118, 134, 150, and 166;
(v) an amino acid sequence with 90% identity to a sequence selected from the group consisting of SEQ ID NOs: 6, 22, 38, 54, 70, 86, 102, 118, 134, 150, and 166; or
(vi) an amino acid sequence with 3, 2 or 1 amino acid difference to a sequence selected from the group consisting of SEQ ID NOs: 6, 22, 38, 54, 70, 86, 102, 118, 134, 150, and 166; and

(c) CDR3 comprises:
(vii) an amino acid sequence selected from the group consisting of SEQ ID NOs: 8, 24, 40, 56, 72, 88, 104, 120, 136, 152, and 168;
(viii) an amino acid sequence with 90% identity to a sequence selected from the group consisting of SEQ ID NOs: 8, 24, 40, 56, 72, 88, 104, 120, 136, 152, and 168; or
(ix) an amino acid sequence with 3, 2 or 1 amino acid difference to a sequence selected from the group consisting of SEQ ID NOs: 8, 24, 40, 56, 72, 88, 104, 120, 136, 152, and 168.

In certain further embodiments, the antigen-binding protein further comprises a second immunoglobulin variable domain comprising three CDRs (CDR4, CDR5 and CDR6), wherein:

(a) CDR4 comprises:
(i) an amino acid sequence selected from the group consisting of SEQ ID NOs: 12, 28, 44, 60, 76, 92, 108, 124, 140, 156, and 172;
(ii) an amino acid sequence with 90% identity to a sequence selected from the group consisting of SEQ ID NOs: 12, 28, 44, 60, 76, 92, 108, 124, 140, 156, and 172; or
(iii) an amino acid sequence with 3, 2 or 1 amino acid difference to a sequence selected from the group consisting of SEQ ID NOs: 12, 28, 44, 60, 76, 92, 108, 124, 140, 156, and 172;

(b) CDR5 comprises:
(iv) an amino acid sequence selected from the group consisting of SEQ ID NOs: 14, 30, 46, 62, 78, 94, 110, 126, 142, 158, and 174;
(v) an amino acid sequence with 90% identity to a sequence selected from the group consisting of SEQ ID NOs: 14, 30, 46, 62, 78, 94, 110, 126, 142, 158, and 174; or
(vi) an amino acid sequence with 3, 2 or 1 amino acid difference to a sequence selected from the group consisting of SEQ ID NOs: 14, 30, 46, 62, 78, 94, 110, 126, 142, 158, and 174; and

(c) CDR6 comprises:
(vii) an amino acid sequence selected from the group consisting of SEQ ID NOs: 16, 32, 48, 64, 80, 96, 112, 128, 144, 160, and 176;
(viii) an amino acid sequence with 90% identity to a sequence selected from the group consisting of SEQ ID NOs: 16, 32, 48, 64, 80, 96, 112, 128, 144, 160, and 176; or
(ix) an amino acid sequence with 3, 2 or 1 amino acid difference to a sequence selected from the group consisting of SEQ ID NOs: 16, 32, 48, 64, 80, 96, 112, 128, 144, 160, and 176.

The present invention also provides antigen-binding proteins, comprising a CDR3 and a CDR6 amino acid sequence pair (CDR3/CDR6) comprising any of the CDR3 amino acid sequences listed in Table 1 paired with any of the CDR6 amino acid sequences listed in Table 1. According to certain embodiments, the present invention provides antigen-binding proteins, comprising an HCDR3/LCDR3 amino acid sequence pair contained within any of the exemplary anti-MBG antigen-binding proteins listed in Table 1. In certain embodiments, the CDR3/CDR6 amino acid sequence pair is selected from the group consisting of SEQ ID NOs: 8/16 (e.g., H4H14357P), 56/64 (e.g., H4H14371P), and 104/112 (e.g., H4H14401P).

The present invention also provides antigen-binding proteins, comprising a set of six CDRs (i.e., CDR1-CDR2-CDR3-CDR4-CDR5-CDR6) contained within any of the exemplary anti-MBG antigen-binding proteins listed in Table 1. In certain embodiments, the CDR1-CDR2-CDR3-CDR4-CDR5-CDR6 amino acid sequence set is selected from the group consisting of SEQ ID NOs: 4-6-8-12-14-16 (e.g., H4H14357P), 52-54-56-60-62-64 (e.g., H4H14371P); and 100-102-104-108-110-112 (e.g., H4H14401P).

In a related embodiment, the present invention provides antigen-binding proteins, comprising a set of six CDRs (i.e., CDR1-CDR2-CDR3-CDR4-CDR5-CDR6) contained within a VR1/VR2 amino acid sequence pair as defined by any of the exemplary anti-MBG antigen-binding proteins listed in Table 1. For example, the present invention includes antigen-binding proteins, comprising the CDR1-CDR2-CDR3-CDR4-CDR5-CDR6 amino acid sequences set contained within a VR1/VR2 amino acid sequence pair selected from the group consisting of SEQ ID NOs: 2/10 (e.g., H4H14357P), 50/58 (e.g., H4H14371P); and 98/106 (e.g., H4H14401P). Methods and techniques for identifying CDRs within VR amino acid sequences are well known in the art and can be used to identify CDRs within the specified VR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within the antigen-binding domain of an antigen-binding protein or an antibody.

The present invention includes anti-MBG antigen-binding proteins comprising a Fc domain, wherein the Fc domain comprises IgG1 or IgG4 isotype as described elsewhere herein. In certain embodiments, the Fc domain comprises an amino acid sequence selected from the group consisting of SEQ ID NOs: 183, 184, 185, 186 and 187.

The present invention includes anti-MBG antigen-binding proteins having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

The present invention also provides for antigen-binding proteins that compete for specific binding to MBG with an antigen-binding protein comprising the CDRs of VR1 and the CDRs of VR2, wherein the VR1 and VR2 each has an amino acid sequence selected from the VR1 and VR2 sequences listed in Table 1.

The present invention also provides antigen-binding proteins that cross-compete for binding to MBG with a reference antigen-binding protein comprising the CDRs of VR1 and the CDRs of VR2, wherein the VR1 and VR2 each has an amino acid sequence selected from the VR1 and VR2 sequences listed in Table 1.

In some embodiments, the antigen-binding protein may bind specifically to MBG in an agonist manner, i.e., it may enhance or stimulate MBG binding and/or activity; in other embodiments, the antigen-binding protein may bind specifically to MBG in an antagonist manner, i.e., it may block MBG from binding to Na+/K+ ATPase.

In one embodiment, the invention provides an isolated antigen-binding protein that has one or more of the following characteristics: (a) comprises an antigen-binding and a Fc domain; (b) is fully human; (c) binds to MBG with a dissociation constant (K_{D}) of less than 100 nM, as measured in a Isothermal titration calorimetry assay; (d) blocks binding of MBG to Na+/K+ ATPase; (e) neutralizes MBG inhibition of membrane repolarization with an EC₅₀ less than 300 nM, less than 200 nM, less than 150 nM or less than 100 nM, as measured in a membrane potential assay; (e) binds to one or more glycosides selected from the group consisting of ouabain, bufalin, cinobufagin, cinobufotalin, resibufagenin, telcinobufagin, 19-norbufalin, proscillaridin, and neriifolin; and (f) does not bind to digitalis or digoxin.

In a related aspect, the present invention provides an antigen-binding protein or antigen-binding fragment thereof that specifically binds MBG, comprising a first immunoglobulin variable domain comprising three CDRs (CDR1, CDR2 and CDR3) and a second immunoglobulin variable domain comprising three CDRs (CDR4, CDR5 and CDR6), wherein CDR1 comprises an amino acid sequence of the formula X¹-X²-X³-X⁴-X⁵-X⁶-X⁷-X⁸-X⁹-X¹⁰-X¹¹-X¹² (SEQ ID NO: 177), wherein X¹=Gln, X²=Ser or Asn, X³=Val, or Ile, X⁴=Leu, Ser, Asn or Gly, X⁵=Tyr, Asn or Ser, X⁶=Ser, Trp, Arg or Asn, X⁷=Ser or absent, X⁸=Asn or absent, X⁹=Asn or absent, X¹⁰=Lys or absent, X¹¹=Asn or absent, and X¹²=Tyr or absent; CDR2 comprises an amino acid sequence of the formula X¹—X²-X³ (SEQ ID NO: 178), wherein X¹=Lys, Gly, Gln or Trp, X²=Ala, and X³=Ser; CDR3 comprises an amino acid sequence of the formula X¹-X²-X³-X⁴-X⁵-X⁶-X⁷-X⁸-X⁹-X¹⁰-X¹¹-X¹² (SEQ ID NO: 179), wherein X¹=Gln, X²=Gln, Glu or His, X³=Tyr or Phe, X⁴=Phe, Tyr or Trp, X⁵=Lys, Ser, Thr or Gly, X⁶=Trp, Thr, Ala or Ile, X⁷=Pro, Leu or absent, X⁸=Arg, Pro, Trp or absent, X⁹=Gly, Thr or absent, X¹⁰=Lys, Trp or absent, X¹¹=Thr, Trp or absent, and X¹²=Thr or absent; CDR4 comprises an amino acid sequence of the formula X¹-X²-X³-X⁴-X⁵-X⁶-X⁷ (SEQ ID NO: 180), wherein X¹=Gln, X²=Ser or Asn, X³=Val or Ile, X⁴=Ser, Arg or Gly, X⁵=Ser, Phe, Arg or Asn, X⁶=Ser, Asn or Tyr, and X⁷=Tyr or absent; CDR5 comprises an amino acid sequence of the formula X¹—X²-X³ (SEQ ID NO: 181), wherein X¹=Asp, Val, Gly or Ala, X²=Ala, and X³=Ser; and CDR6 comprises an amino acid sequence of the formula X¹-X²-X³-X⁴-X⁵-X⁶-X⁷-X⁸-X⁹ (SEQ ID NO: 182), wherein X¹=Gln, X²=Gln, X³=Tyr or Ser, X⁴=Gly, Tyr, Ser or Ile, X⁵=Ser or Arg, X⁶=Ser, Asp or Thr, X⁷=Pro, X⁸=Phe, Tyr, Arg or Pro, and X⁹=Thr or Ile. In certain embodiments, the first immunoglobulin variable domain is variable region selected from the group consisting of VR1 and VR2 amino acid sequences listed in Table 1.

In specific embodiments, the present invention provides an antigen-binding protein or antigen-binding fragment thereof that specifically binds MBG, comprising a first immunoglobulin variable domain comprising three CDRs (CDR1, CDR2 and CDR3) and a second immunoglobulin variable domain comprising three CDRs (CDR4, CDR5 and CDR6), wherein CDR1 comprises an amino acid sequence of the formula X¹-X²-X³-X⁴-X⁵-X⁶-X⁷-X⁸-X⁹-X¹⁰-X¹¹-X¹² (SEQ ID NO: 177), wherein X¹=Gln, X²=Ser, X³=Val, or Ile, X⁴=Leu, Ser, or Gly, X⁵=Tyr or Asn, X⁶=Ser or Trp, X⁷=Ser or absent, X⁸=Asn or absent, X⁹=Asn or absent, X¹⁰=Lys or absent, X¹¹=Asn or absent, and X¹²=Tyr or absent; CDR2 comprises an amino acid sequence of the formula X¹-X²-X³-X⁴-X⁵-X⁶-X⁷-X⁸ (SEQ ID NO: 178), wherein X¹=Lys, Gln or Trp, X²=Ala, X³=Ser, X⁴=absent, X⁵=absent, X⁶=absent, X⁷=absent, and X⁸=absent; CDR3 comprises an amino acid sequence of the formula X¹-X²-X³-X⁴-X⁵-X⁶-X⁷-X⁸-X⁹-X¹⁰-X¹¹-X¹²-X¹³-X¹⁴ (SEQ ID NO: 179), wherein X¹=Gln, X²=Gln or His, X³=Tyr, X⁴=Tyr, X⁵=Ser or Gly, X⁶=Ala or Ile, X⁷=Leu or absent, X⁸=Trp or absent, X⁹=Thr or absent, X¹⁰=absent, X¹¹=absent, X¹²=absent, X¹³=absent, and X¹⁴=absent; CDR4 comprises an amino acid sequence of the formula X¹-X²-X³-X⁴-X⁵-X⁶-X⁷ (SEQ ID NO: 180), wherein X¹=Gln, X²=Ser, X³=Val, X⁴=Ser or Gly, X⁵=Ser or Asn, X⁶=Ser or Asn, and X⁷=Tyr; CDR5 comprises an amino acid sequence of the formula X¹—X²-X³ (SEQ ID NO: 181), wherein X¹=Asp or Gly, X²=Ala, and X³=Ser; and CDR6 comprises an amino acid sequence of the formula X¹-X²-X³-X⁴-X⁵-X⁶-X⁷-X⁸-X⁹ (SEQ ID NO: 182), wherein X¹=Gln, X²=Gln, X³=Tyr, X⁴=Gly or Ser, X⁵=Ser or Arg, X⁶=Ser, X⁷=Pro, X⁸=Phe or Tyr, and X⁹=Thr or Ile.

In a second aspect, the present invention provides nucleic acid molecules encoding anti-MBG antigen-binding proteins or portions thereof. For example, the present invention provides nucleic acid molecules encoding any of the VR1 amino acid sequences listed in Table 1; in certain embodiments the nucleic acid molecule comprises a polynucleotide sequence selected from any of the VR1 nucleic acid sequences listed in Table 2, or a substantially similar sequence thereof having at least 90%, at least 95%, at least 98% or at least 99% sequence identity thereto.

The present invention also provides nucleic acid molecules encoding any of the VR2 amino acid sequences listed in Table 1; in certain embodiments the nucleic acid molecule comprises a polynucleotide sequence selected from any of the VR2 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR1 amino acid sequences listed in Table 1; in certain embodiments the nucleic acid molecule comprises a polynucleotide sequence selected from any of the CDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR2 amino acid sequences listed in Table 1; in certain embodiments the nucleic acid molecule comprises a polynucleotide sequence selected from any of the CDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR3 amino acid sequences listed in Table 1; in certain embodiments the nucleic acid molecule comprises a polynucleotide sequence selected from any of the CDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR4 amino acid sequences listed in Table 1; in certain embodiments the nucleic acid molecule comprises a polynucleotide sequence selected from any of the CDR4 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR5 amino acid sequences listed in Table 1; in certain embodiments the nucleic acid molecule comprises a polynucleotide sequence selected from any of the CDR5 nucleic acid sequences listed in Table 2, or a substantially similar sequence thereof having at least 90%, at least 95%, at least 98% or at least 99% sequence identity thereto.

The present invention also provides nucleic acid molecules encoding any of the CDR6 amino acid sequences listed in Table 1; in certain embodiments the nucleic acid molecule comprises a polynucleotide sequence selected from any of the CDR6 nucleic acid sequences listed in Table 2, or a substantially similar sequence thereof having at least 90%, at least 95%, at least 98% or at least 99% sequence identity thereto.

The present invention also provides nucleic acid molecules encoding a VR1, wherein the VR1 comprises a set of three CDRs (i.e., CDR1-CDR2-CDR3), wherein the CDR1-CDR2-CDR3 amino acid sequence set is as defined by any of the exemplary anti-MBG antigen-binding proteins listed in Table 1.

The present invention also provides nucleic acid molecules encoding a VR2, wherein the VR2 comprises a set of three CDRs (i.e., CDR4-CDR5-CDR6), wherein the CDR4-CDR5-CDR6 amino acid sequence set is as defined by any of the exemplary anti-MBG antigen-binding proteins listed in Table 1.

The present invention also provides nucleic acid molecules encoding both a VR1 and a VR2, wherein the VR1 comprises an amino acid sequence of any of the VR1 amino acid sequences listed in Table 1, and wherein the VR2 comprises an amino acid sequence of any of the VR2 amino acid sequences listed in Table 1. In certain embodiments, the nucleic acid molecule comprises a polynucleotide sequence selected from any of the VR1 nucleic acid sequences listed in Table 2, or a substantially similar sequence thereof having at least 90%, at least 95%, at least 98% or at least 99% sequence identity thereto, and a polynucleotide sequence selected from any of the VR2 nucleic acid sequences listed in Table 1, or a substantially similar sequence thereof having at least 90%, at least 95%, at least 98% or at least 99% sequence identity thereto. In certain embodiments according to this aspect of the invention, the nucleic acid molecule encodes a VR1 and VR2, wherein the VR1 and VR2 are both derived from the same anti-MBG antigen-binding proteins listed in Table 1.

In a related aspect, the present invention provides recombinant expression vectors capable of expressing a polypeptide comprising a light chain variable region of an anti-MBG antigen-binding protein. For example, the present invention includes recombinant expression vectors comprising any of the nucleic acid molecules mentioned above, i.e., nucleic acid molecules encoding any of the VR, and/or CDR sequences as set forth in Table 2. Also included within the scope of the present invention are host cells into which such vectors have been introduced, as well as methods of producing the antigen-binding proteins or portions thereof by culturing the host cells under conditions permitting production of the antigen-binding proteins or fragments thereof, and recovering the antigen-binding proteins and fragments so produced.

In a third aspect, the invention provides a pharmaceutical composition comprising a therapeutically effective amount of at least one recombinant antigen-binding protein or antigen-binding fragment thereof which specifically binds MBG and a pharmaceutically acceptable carrier. In a related aspect, the invention features a composition, which is a combination of an anti-MBG antigen-binding protein and a second therapeutic agent. In one embodiment, the second therapeutic agent is any agent that is advantageously combined with an anti-MBG antigen-binding protein. Exemplary agents that may be advantageously combined with an anti-MBG antigen-binding protein include, without limitation, other agents that bind and/or inhibit MBG activity (including other antigen-binding proteins or antigen-binding fragments thereof, etc.) and/or agents which do not directly bind MBG but nonetheless alleviate or ameliorate or treat a MBG-associated disease or disorder (e.g., cardiovascular disease). Additional combination therapies and co-formulations involving the anti-MBG antigen-binding proteins of the present invention are disclosed elsewhere herein.

In a fourth aspect, the invention provides therapeutic methods for treating a disease or disorder associated with MBG such as cardiovascular disease (e.g., hypertension) in a subject using an anti-MBG antigen-binding protein or antigen-binding portion thereof of the invention, wherein the therapeutic methods comprise administering a therapeutically effective amount of a pharmaceutical composition comprising an antigen-binding protein or antigen-binding fragment of an antigen-binding protein of the invention to the subject in need thereof. The disorder treated is any disease or condition which is improved, ameliorated, inhibited or prevented by inhibition of MBG activity. In certain embodiments, the invention provides methods to prevent, treat or ameliorate at least one symptom of a MBG-associated disease or disorder, the method comprising administering a therapeutically effective amount of an anti-MBG antigen-binding protein or antigen-binding fragment thereof of the invention to a subject in need thereof. In some embodiments, the present invention provides methods to ameliorate or reduce the severity of at least one symptom or indication of MBG-associated disease or disorder in a subject by administering a therapeutically effective amount of an anti-MBG antigen-binding protein of the invention, wherein the at least one symptom or indication is selected from the group consisting of atherosclerosis, hypertension, angina, shortness of breath, palpitations in the chest, weakness or dizziness, nausea, sweating, pressure or pain in the chest, arm or below the breastbone, irregular heartbeat, and death. In certain embodiments, the invention provides methods to reduce hypertension in a subject, the methods comprising administering to the subject a therapeutically effective amount of an antigen-binding protein or fragment thereof of the invention that binds MBG and blocks MBG binding to Na+/K+ ATPase. In some embodiments, the antigen-binding protein or antigen-binding fragment thereof may be administered prophylactically or therapeutically to a subject having or at risk of having cardiovascular disease. The subjects at risk include, but are not limited to, an immunocompromised person, subjects of advanced age, pregnant women, and subjects with one or more risk factors including obesity, high blood cholesterol, smoking, excessive alcohol consumption, lack of exercise, and/or diabetes. In certain embodiments, the antigen-binding protein or antigen-binding fragment thereof of the invention is administered in combination with a second therapeutic agent to the subject in need thereof. The second therapeutic agent may be selected from the group consisting of an anti-hypertensive drug (e.g., an angiotensin-converting enzyme inhibitor, an angiotensin receptor blocker, a diuretic, a calcium channel blocker, an alpha-adrenoceptor blocker, an endothelin-1 receptor blocker, an organic nitrate, and a protein kinase C inhibitor), a statin, aspirin, a different antibody or antigen-binding protein to MBG, an inhibitor of ouabain or another cardiac glycoside, a dietary supplement such as anti-oxidants and any other drug or therapy known in the art. In certain embodiments, the second therapeutic agent may be an agent that helps to counteract or reduce any possible side effect(s) associated with an antigen-binding protein or antigen-binding fragment thereof of the invention, if such side effect(s) should occur. The antigen-binding protein or fragment thereof may be administered subcutaneously, intravenously, intradermally, intraperitoneally, orally, intramuscularly, or intracranially. The antigen-binding protein or fragment thereof may be administered at a dose of about 0.1 mg/kg of body weight to about 100 mg/kg of body weight of the subject. In certain embodiments, an antigen-binding protein of the present invention may be administered at one or more doses comprising between 10 mg to 600 mg.

The present invention also includes use of an anti-MBG antigen-binding protein or antigen-binding fragment thereof of the invention in the manufacture of a medicament for the treatment of a disease or disorder that would benefit from the blockade of MBG binding and/or activity.

Other embodiments will become apparent from a review of the ensuing detailed description.

### BRIEF DESCRIPTION OF THE FIGURES

FIG. 1 shows pharmacokinetic profiles of anti-MBG antigen-binding proteins H4H14401P, H4H14371P, H4H14357P and an isotype control antibody in C57BL/6 mice (as described in Example 9 herein).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, preferred methods and materials are now described. All publications mentioned herein are incorporated herein by reference in their entirety.

### Definitions

The term “marinobufagenin”, also called as “MBG”, or “3β, 5β-dihydroxy-14,15-epoxybufadienolide”, refers to the endogenous cardiac glycoside, synthesized from cholesterol, primarily by the adrenal gland. Like the other cardiac glycosides, MBG induces vasoconstriction and acts as a cardiac inotrope. It circulates at plasma concentrations of <1 nM and binds and inhibits Na+/K+ ATPase, resulting in increased intracellular sodium. Elevated intracellular sodium alters the sodium calcium exchanger pump, resulting in increased intracellular calcium levels and as such, enhanced force of smooth muscle contraction (hypertension) and enhanced cardiac contractility (inotropy).

The term “Na+/K+ ATPase”, also known as “sodium-potassium adenosine triphosphatase” or “sodium-potassium pump” or “sodium pump” refers to the transmembrane ATPase located in the plasma membrane of all animal cells. The enzyme pumps sodium out of cells, while pumping potassium into cells. Na+/K+ ATPase consists of three subunits: α, β and FXYD. There are 4α isoforms with varying tissue expression: α1 expressed abundantly in most tissues (highest expression in kidneys), α2 expressed in brain, heart, skeletal and vascular smooth muscle, and adipocytes, α3 expressed in neurons and ovaries, and α4 expressed in sperm. Na+/K+ ATPase helps maintain resting potential, effect transport, and regulate cellular volume. It also functions as a signal transducer/integrator to regulate MAPK pathway, reactive oxygen species, as well as intracellular calcium. Inhibition of Na+/K+ ATPase by MBG or other cardiac glycosides leads to two major effects: (i) increased intracellular Na+ and Ca+ resulting in enhanced muscle contraction and/or alteration in renal Na transport; and (ii) stimulation of downstream signaling via associated signaling proteins. Ultimately, this results in inotropy, hypertension, increased cell proliferation and fibrosis. Unless specified as being from a non-human species, the term “Na+/K+ ATPase”, as used herein, means human Na+/K+ ATPase.

The term “antigen-binding protein”, as used herein, is intended to refer to immunoglobulin molecules comprised of four polypeptide chains, two heavy (H) chains and two light (L) chains inter-connected by disulfide bonds, as well as multimers thereof (e.g. IgM) or antigen-binding fragments thereof. Each heavy chain is comprised of a heavy chain constant region (comprised of domains C_{H}1, C_{H}2 and C_{H}3) and an Ig variable region which may be a heavy chain variable region (“HCVR” or “V_{H}”) or a light chain variable region (“LCVR or “V_{L}”). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs. The term “antigen-binding protein”, as used herein, also includes antibodies.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-MBG monoclonal antigen-binding proteins disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antigen-binding proteins, and antigen-binding fragments thereof, which are derived from any of the amino acid sequences disclosed herein, wherein one or more amino acids within one or more framework and/or CDR regions are mutated to the corresponding residue(s) of the germline sequence from which the antigen-binding protein was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding protein was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding protein was originally derived). Furthermore, the antigen-binding proteins of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding proteins and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antigen-binding proteins and antigen-binding fragments obtained in this general manner are encompassed within the present invention.

The present invention also includes fully human anti-MBG monoclonal antigen-binding proteins comprising variants of any of the VR, and/or CDR amino acid sequences disclosed herein having one or more conservative substitutions. For example, the present invention includes anti-MBG antigen-binding proteins having VR, and/or CDR amino acid sequences with, e.g., 10 or fewer, 8 or fewer, 6 or fewer, 4 or fewer, etc. conservative amino acid substitutions relative to any of the VR, and/or CDR amino acid sequences disclosed herein.

The terms “fully human antibody”, “human antibody”, “fully human antigen-binding protein”, or “human antigen-binding protein”, as used herein, are intended to include antigen-binding proteins having variable and constant regions derived from human germline immunoglobulin sequences. The human antigen-binding proteins of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antigen-binding protein”, as used herein, is not intended to include antigen-binding proteins in which CDR sequences derived from the germline of another mammalian species (e.g., mouse), have been grafted onto human FR sequences. The term includes antigen-binding proteins or antibodies recombinantly produced in a non-human mammal, or in cells of a non-human mammal. The term is not intended to include antigen-binding proteins or antibodies isolated from or generated in a human subject.

The term “recombinant”, as used herein, refers to antigen-binding proteins or antigen-binding fragments thereof of the invention created, expressed, isolated or obtained by technologies or methods known in the art as recombinant DNA technology which include, e.g., DNA splicing and transgenic expression. The term refers to antigen-binding proteins expressed in a non-human mammal (including transgenic non-human mammals, e.g., transgenic mice), or a cell (e.g., CHO cells) expression system or isolated from a recombinant combinatorial human antibody library.

The term “specifically binds,” or “binds specifically to”, or the like, means that an antigen-binding protein or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, isothermal titration calorimetry, and the like. As described herein, antigen-binding proteins have been identified by isothermal titration calorimetry, which bind specifically to MBG. Moreover, multi-specific antigen-binding proteins that bind to one epitope in MBG and one or more additional antigens or a bi-specific that binds to two different regions of MBG are nonetheless considered antigen-binding proteins that “specifically bind”, as used herein.

The term “high affinity” antigen-binding protein refers to those antigen-binding proteins having a binding affinity to MBG, expressed as K_{D}, of at least 10⁻⁸ M; preferably 10⁻⁹ M; more preferably 10⁻¹⁰M, even more preferably 10⁻¹¹ M, even more preferably 10⁻¹² M, as measured by isothermal titration calorimetry or solution-affinity ELISA.

By the term “slow off rate”, “Koff” or “kd” is meant an antigen-binding protein that dissociates from MBG, with a rate constant of 1×10⁻³ s⁻¹ or less, preferably 1×10⁻⁴ s⁻¹ or less, as determined by isothermal titration calorimetry.

The terms “antigen-binding portion” of an antigen-binding protein, “antigen-binding fragment” of an antigen-binding protein, and the like, as used herein, include any naturally occurring, enzymatically obtainable, synthetic, or genetically engineered polypeptide or glycoprotein that specifically binds an antigen to form a complex. The terms “antigen-binding fragment” of an antigen-binding protein or antibody, or “antibody fragment”, as used herein, refers to one or more fragments of an antigen-binding protein that retain the ability to bind to MBG.

In specific embodiments, antigen-binding protein or antigen-binding fragments thereof may be conjugated to a moiety such a ligand or a therapeutic moiety (“immunoconjugate”), such as an anti-hypertensive drug, a second anti-MBG antigen-binding protein, or any other therapeutic moiety useful for treating a disease or disorder associated with MBG.

An “isolated antigen-binding protein”, as used herein, is intended to refer to an antigen-binding protein that is substantially free of other antigen-binding proteins having different antigenic specificities (e.g., an isolated antigen-binding protein that specifically binds MBG, or a fragment thereof, is substantially free of other antigen-binding proteins that specifically bind antigens other than MBG.

A “blocking antigen-binding protein” or a “neutralizing antigen-binding protein “, as used herein (or an” antigen-binding protein that neutralizes MBG activity” or “antagonist antigen-binding protein”), is intended to refer to an antigen-binding protein whose binding to MBG results in inhibition of at least one biological activity of MBG. For example, an antigen-binding protein of the invention may prevent or block MBG binding to Na+/K+ ATPase.

The term “isothermal titration calorimetry”, as used herein, refers to a physical phenomenon that allows for the analysis of thermodynamic parameters of real-time biomolecular interactions with small molecules in solution, for example using the MicroCal™ Auto-iTC₂₀₀ system (GE Healthcare).

The term “K_{D}”, as used herein, is intended to refer to the equilibrium dissociation constant of a particular antigen-binding protein-antigen interaction.

The term “cross-competes”, as used herein, means an antigen-binding protein or antigen-binding fragment thereof binds to an antigen and inhibits or blocks the binding of another antigen-binding protein or antigen-binding fragment thereof. The term also includes competition between two antigen-binding proteins in both orientations, i.e., a first antigen-binding protein that binds and blocks binding of second antigen-binding protein and vice-versa. In certain embodiments, the first antigen-binding protein and second antigen-binding protein may bind to the same epitope. Alternatively, the first and second antigen-binding proteins may bind to different, but overlapping epitopes such that binding of one inhibits or blocks the binding of the second antigen-binding protein, e.g., via steric hindrance. Cross-competition between antigen-binding proteins may be measured by methods known in the art, for example, by a real-time, label-free bio-layer interferometry assay. Cross-competition between two antigen-binding proteins may be expressed as the binding of the second antigen-binding protein that is less than the background signal due to self-self binding (wherein first and second antigen-binding proteins is the same antigen-binding protein). Cross-competition between 2 antigen-binding proteins may be expressed, for example, as % binding of the second antigen-binding protein that is less than the baseline self-self background binding (wherein first and second antigen-binding proteins is the same antigen-binding protein).

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, more preferably a human, in need of amelioration, prevention and/or treatment of a disease or disorder associated with MBG. The term includes human subjects who have or are at risk of having a disease or disorder associated with MBG.

As used herein, the terms “treat”, “treating”, or “treatment” refer to the reduction or amelioration of the severity of at least one symptom or indication of a disease or disorder associated with MBG due to the administration of a therapeutic agent such as an antigen-binding protein of the present invention to a subject in need thereof. The terms include inhibition of progression of disease or of worsening of symptoms. The terms also include positive prognosis of disease, i.e., the subject may be free of a symptom or indication or may have reduced intensity of a symptom or indication upon administration of a therapeutic agent such as an antigen-binding protein of the present invention. For example, a subject with hypertension or cardiovascular disease may have reduction in systolic and/or diastolic blood pressure upon administration of an antigen-binding protein of the invention. The therapeutic agent may be administered at a therapeutic dose to the subject.

The terms “prevent”, “preventing” or “prevention” refer to inhibition of manifestation of any symptoms or indications of a disease or disorder associated with MBG upon administration of an antigen-binding protein of the present invention. The term includes inhibition of manifestation of a symptom or indication of a MBG-associated disease or disorder in a subject at risk for developing such a disease or disorder.

The inventors have described herein fully human antigen-binding proteins and antigen-binding fragments thereof that specifically bind to MBG and modulate the interaction of MBG with Na+/K+ ATPase. Prior to the present invention, there were no fully human antigen-binding proteins, including antibodies that specifically bound to MBG with high affinity and blocked its activity. Accordingly, the present invention discloses fully human antigen-binding proteins comprising an antigen-binding domain and a Fc domain. In certain embodiments, the antigen-binding domain comprises a first and second immunoglobulin light chain variable region comprising CDRs selected from: (a) CDR sequences listed in Table 1; (b) CDR sequences with 90% identity to sequences listed in Table 1; or (c) CDR sequences with 95% identity to sequences listed in Table 1.

The anti-MBG antigen-binding proteins of the present invention bind to MBG with high affinity. In certain embodiments, the antigen-binding proteins of the present invention are blocking antigen-binding proteins wherein the antigen-binding proteins may bind to MBG and block the interaction of MBG with Na+/K+ ATPase. In some embodiments, the blocking antigen-binding proteins of the invention may block the binding of MBG to Na+/K+ ATPase and/or neutralize MBG inhibition of membrane repolarization. In some embodiments, the blocking antigen-binding proteins may be useful for treating a subject suffering from cardiovascular disease. The antigen-binding proteins when administered to a subject in need thereof may reduce hypertension in the subject. They may be used to improve the outcome of pre-eclampsia or extend pregnancy in a subject. They may be used alone or as adjunct therapy with other therapeutic moieties or modalities known in the art for treating cardiovascular disease.

Certain anti-MBG antigen-binding proteins of the present invention are able to bind to and neutralize the activity of MBG, as determined by in vitro or in vivo assays. The ability of the antigen-binding proteins of the invention to bind to and neutralize the activity of MBG may be measured using any standard method known to those skilled in the art, including binding assays, or activity assays, as described herein.

Non-limiting, exemplary in vitro assays for measuring binding are illustrated in Examples 3-4, herein. In Examples 3 and 4, the binding affinity and dissociation constants of anti-MBG antigen-binding proteins for MBG were determined by isothermal titration calorimetry assay and by surface plasmon resonance, respectively. Examples 5 and 6 describe isoelectric point and thermal stability of the anti-MBG antigen-binding proteins. In Example 7, membrane potential assays were used to determine inhibition of MBG activity in membrane repolarization. Examples 8 and 9 describe the in vivo efficacy and pharmacokinetics, respectively, of the anti-MBG antigen-binding proteins. Examples 10 and 12 describe solubility, viscosity and stability of anti-MBG proteins in solution. Example 12 describes the crystallization of an exemplary anti-MBG antigen-binding protein.

The antigen-binding proteins specific for MBG may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antigen-binding proteins may be used in diagnostic assays including imaging assays.

### Antigen-Binding Fragments of Antigen-Binding Proteins

The terms “antigen-binding portion” of an antigen-binding protein, “antigen-binding fragment” of an antigen-binding protein, and the like, as used herein, include any naturally occurring, enzymatically obtainable, synthetic, or genetically engineered polypeptide or glycoprotein that specifically binds an antigen to form a complex. The terms “antigen-binding fragment” of an antigen-binding protein, or “antigen-binding protein fragment”, as used herein, refers to one or more fragments of an antigen-binding protein that retain the ability to specifically bind to MBG. An antigen-binding protein fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antigen-binding protein or an antibody may be derived, e.g., from full protein molecules using any suitable standard techniques such as proteolytic digestion or recombinant genetic engineering techniques involving the manipulation and expression of DNA encoding antigen-binding protein variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antigen-binding protein or an antibody of the present invention will typically comprise at least one immunoglobulin (Ig) variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antigen-binding protein may contain a monomeric V_{H} or V_{L} domain.

In certain embodiments, an antigen-binding fragment may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; V_{H}—C_{L}; V_{L}-C_{H}1; (ix) -C_{H}2; (x) V_{L}-C_{H}3; (xi) -C_{H}1-C_{H}2; (xii) V_{L}-C_{H}1-C_{H}2-C_{H}3, (xiii) V_{L}-C_{H}2-C_{H}3; and (xiv) -C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antigen-binding protein of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full protein molecules, antigen-binding fragments may be mono-specific or multi-specific (e.g., bi-specific). A multi-specific antigen-binding fragment of an antigen-binding protein will typically comprise at least two different variable domains, wherein each variable domain is capable of specifically binding to a separate antigen or to a different epitope on the same antigen. Any multi-specific antigen-binding protein format, including the exemplary bi-specific antigen-binding protein formats disclosed herein, may be adapted for use in the context of an antigen-binding fragment of an antigen-binding protein of the present invention using routine techniques available in the art.

### Preparation of Human Antigen-Binding Proteins

Methods for generating human antigen-binding proteins (including antibodies) in transgenic mice are known in the art. Any such known methods can be used in the context of the present invention to make human antigen-binding proteins that specifically bind to MBG.

Using VELOCIMMUNE® technology (see, for example, U.S. Pat. No. 6,596,541, Regeneron Pharmaceuticals, VELOCIMMUNE®) or any other known method for generating monoclonal antigen-binding proteins, high affinity chimeric antigen-binding proteins to MBG are initially isolated having a human variable region and a mouse constant region. The VELOCIMMUNE® technology involves generation of a transgenic mouse having a genome comprising human Ig variable regions (heavy and/or light chain) operably linked to endogenous mouse constant region loci such that the mouse produces an antibody or antigen-binding protein comprising a human variable region and a mouse constant region in response to antigenic stimulation. The DNA encoding the variable regions (heavy and/or light chains) of the antigen-binding protein are isolated and operably linked to DNA encoding the human heavy and light chain constant regions. The DNA is then expressed in a cell capable of expressing the fully human antigen-binding protein.

Generally, a VELOCIMMUNE® mouse is challenged with the antigen of interest, and lymphatic cells (such as B-cells) are recovered from the mice that express antigen-binding proteins. The lymphatic cells may be fused with a myeloma cell line to prepare immortal hybridoma cell lines, and such hybridoma cell lines are screened and selected to identify hybridoma cell lines that produce antigen-binding proteins specific to the antigen of interest. DNA encoding the variable regions (heavy chain and/or light chain) may be isolated and linked to desirable isotypic constant regions of the heavy chain and light chain. Such an antigen-binding protein may be produced in a cell, such as a CHO cell. Alternatively, DNA encoding the antigen-specific chimeric antigen-binding proteins or the variable domains of the light chains may be isolated directly from antigen-specific lymphocytes.

Initially, high affinity chimeric antigen-binding proteins are isolated having a human variable region and a mouse constant region. As in the experimental section below, the antigen-binding proteins are characterized and selected for desirable characteristics, including affinity, selectivity, epitope, etc. The mouse constant regions are replaced with a desired human constant region to generate the fully human antigen-binding protein of the invention, for example wild-type or modified IgG1 or IgG4. While the constant region selected may vary according to specific use, high affinity antigen-binding and target specificity characteristics reside in the variable region.

### Bioequivalents

The anti-MBG antigen-binding proteins and fragments thereof of the present invention encompass proteins having amino acid sequences that vary from those of the described antigen-binding proteins, but that retain the ability to bind MBG. Such variant antigen-binding proteins and fragments thereof comprise one or more additions, deletions, or substitutions of amino acids when compared to parent sequence, but exhibit biological activity that is essentially equivalent to that of the described antigen-binding proteins. Likewise, the antigen-binding protein-encoding DNA sequences of the present invention encompass sequences that comprise one or more additions, deletions, or substitutions of nucleotides when compared to the disclosed sequence, but that encode an antigen-binding protein or fragment thereof that is essentially bioequivalent to an antigen-binding protein or fragment thereof of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antigen-binding proteins or antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antigen-binding protein or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antigen-binding protein (or its target) is measured as a function of time; and (d) in a well-controlled clinical trial that establishes safety, efficacy, or bioavailability or bioequivalence of an antigen-binding protein.

Bioequivalent variants of the antigen-binding proteins of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins or antibodies may include variants comprising amino acid changes, which modify the glycosylation characteristics of the antigen-binding proteins or antibodies, e.g., mutations that eliminate or remove glycosylation.

### Anti-MBG Antigen-Binding Proteins Comprising Fc Variants

According to certain embodiments of the present invention, anti-MBG antigen-binding proteins are provided comprising an Fc domain comprising one or more mutations which enhance or diminish antigen-binding protein binding to the FcRn receptor, e.g., at acidic pH as compared to neutral pH. For example, the present invention includes anti-MBG antigen-binding proteins comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antigen-binding protein when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-MBG antigen-binding proteins comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 2571 and 3111 (e.g., P2571 and 03111); 2571 and 434H (e.g., P2571 and N434H); 376V and 434H (e.g., D376V and N434H); 307A, 380A and 434A (e.g., 1307A, E380A and N434A); and 433K and 434F (e.g., H433K and N434F). All possible combinations of the foregoing Fc domain mutations and other mutations within the Ig variable domains disclosed herein, are contemplated within the scope of the present invention.

The present invention also includes anti-MBG antigen-binding proteins comprising a chimeric heavy chain constant (C_{H}) region, wherein the chimeric C_{H} region comprises segments derived from the C_{H} regions of more than one immunoglobulin isotype. For example, the antibodies of the invention may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the invention comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antigen-binding protein comprising a chimeric C_{H} region as described herein may, in certain embodiments, exhibit modified Fc effector functions without adversely affecting the therapeutic or pharmacokinetic properties of the antigen-binding protein (See, e.g., U.S. Patent Publication US2014/0243504, the disclosure of which is hereby incorporated by reference in its entirety).

### Biological Characteristics of the Antigen-Binding Proteins

In general, the antigen-binding proteins of the present invention function by binding to MBG. In certain embodiments, the antigen-binding proteins of the present invention bind with high affinity to MBG. For example, the present invention includes antigen-binding proteins and antigen-binding fragments thereof that bind MBG (e.g., at 25° C. or at 37° C.) with a K_{D} of less than 870 nM as measured by isothermal titration calorimetry, e.g., using the assay format as defined in Example 3 herein. In certain embodiments, the antigen-binding proteins or antigen-binding fragments thereof bind MBG with a K_{D} of of less than 870 nM, less than 700 nM, less than 500 nM, less than 250 nM, less than 100 nM, less than 50 nM, or less than 25 nM, as measured by isothermal titration calorimetry, e.g., using the assay format as defined in Example 3 herein, or a substantially similar assay.

The present invention also includes antigen-binding proteins or antigen-binding fragments thereof that neutralize or block MBG inhibition of membrane repolarization. In certain embodiments, the antigen-binding proteins block MBG binding to Na+/K+ ATPase pump and repress MBG inhibition of membrane repolarization. In some embodiments, the antigen-binding proteins inhibited MBG activity and facilitated membrane repolarization with EC₅₀ less than 300 nM, less than 200 nM, less than 150 nM or less than 100 nM in a membrane potential assay, e.g., as shown in Example 4, or a substantially similar assay.

In certain embodiments, the antigen-binding proteins of the present invention may function by blocking or inhibiting the Na+/K+ ATPase-binding activity associated with MBG. In certain embodiments, the antigen-binding proteins of the invention may cross-react with one or more glycosides selected from the group consisting of ouabain, bufalin, cinobufagin, cinobufotalin, resibufagenin, telcinobufagin, 19-norbufalin, proscillaridin, and neriifolin. In some embodiments, the antigen-binding proteins of the invention do not cross-react with digitalis or digoxin.

In certain embodiments, the antigen-binding proteins of the present invention may be bi-specific antigen-binding proteins. The bi-specific antigen-binding proteins of the invention may bind one epitope and may also bind a second epitope of MBG. In certain embodiments, the bi-specific antigen-binding proteins of the invention may bind MBG and another cardiac glycoside.

In one embodiment, the invention provides an isolated recombinant antigen-binding protein or antigen-binding fragment thereof that binds specifically to MBG, wherein the antigen-binding protein or fragment thereof exhibits one or more of the following characteristics: (a) comprises an antigen-binding domain and a Fc domain; (b) is fully human; (c) binds to MBG with a dissociation constant (K_{D}) of less than 800 nM, as measured in an isothermal titration calorimetry assay; (d) blocks binding of MBG to Na+/K+ ATPase; (e) neutralizes MBG inhibition of membrane repolarization with an EC₅₀ less than 300 nM, less than 200 nM, less than 150 nM or less than 100 nM, as measured in a membrane potential assay; (e) binds to one or more glycosides selected from the group consisting of ouabain, bufalin, cinobufagin, cinobufotalin, resibufagenin, telcinobufagin, 19-norbufalin, proscillaridin, and neriifolin; and (f) does not bind to digitalis or digoxin.

In certain embodiments, the invention provides an isolated recombinant antigen-binding protein or antigen-binding fragment thereof that binds specifically to MBG, wherein the antigen-binding protein or fragment thereof exhibits one or more of the following characteristics: (a) binds to MBG with a dissociation constant (K_{D}) of less than 25 nM, as measured in a isothermal titration calorimetry assay at 25° C.; (b) binds to MBG with a dissociation constant (K_{D}) of less than 10 nM, as measured in a surface plasmon resonance assay at 25° C.; (c) blocks binding of MBG to Na+/K+ ATPase; (d) releases inhibition of Na+/K+ ATPase and facilitates membrane repolarization of a cell with EC₅₀ less than 300 nM, less than 200 nM, less than 150 nM or less than 100 nM, as measured in a membrane potential assay; (e) does not bind to digitalis or digoxin; (f) is fully human; and (g) the antigen-binding domain comprises at least one immunoglobulin variable region comprising three complementarity determining regions (CDRs), as set in Table 1.

The antigen-binding proteins of the present invention may possess one or more of the aforementioned biological characteristics, or any combinations thereof. Other biological characteristics of the antigen-binding proteins of the present invention will be evident to a person of ordinary skill in the art from a review of the present disclosure including the working Examples herein.

### Immunoconjugates

The invention encompasses a human anti-MBG monoclonal antigen-binding protein conjugated to a therapeutic moiety (“immunoconjugate”), such as an anti-hypertensive drug to treat cardiovascular disease. As used herein, the term “immunoconjugate” refers to an antigen-binding protein which is chemically or biologically linked to a radioactive agent, a cytokine, an interferon, a target or reporter moiety, an enzyme, a peptide or protein or a therapeutic agent. The antigen-binding protein may be linked to the radioactive agent, cytokine, interferon, target or reporter moiety, enzyme, peptide or therapeutic agent at any location along the molecule so long as it is able to bind its target. Examples of immunoconjugates include antibody drug conjugates and antibody-toxin fusion proteins. In one embodiment, the agent may be a second different antigen-binding protein to MBG or another cardiac glycoside. The type of therapeutic moiety that may be conjugated to the anti-MBG antigen-binding protein and will take into account the condition to be treated and the desired therapeutic effect to be achieved. Examples of suitable agents for forming immunoconjugates are known in the art; see for example, WO 05/103081.

### Multi-Specific Antigen-Binding Proteins

The antigen-binding proteins of the present invention may be mono-specific, bi-specific, or multi-specific. Multi-specific antigen-binding proteins may be specific for different epitopes of the target molecule. See, e.g., Tutt et al., 1991, J. Immunol. 147:60-69; Kufer et al., 2004, Trends Biotechnol. 22:238-244.

Any of the multi-specific antigen-binding molecules of the invention, or variants thereof, may be constructed using standard molecular biological techniques (e.g., recombinant DNA and protein expression technology), as will be known to a person of ordinary skill in the art.

In some embodiments, MBG-specific antigen-binding proteins are generated in a bi-specific format (a “bi-specific”) in which variable regions binding to distinct domains of MBG are linked together to confer dual-domain specificity within a single binding molecule. Appropriately designed bi-specifics may enhance overall MBG inhibitory efficacy through increasing both specificity and binding avidity. Variable regions with specificity for individual domains, or that can bind to different regions within one domain, are paired on a structural scaffold that allows each region to bind simultaneously to the separate epitopes, or to different regions within one domain. In one example for a bi-specific, heavy chain variable regions (V_{H}) or light chain variable regions (V_{L}) from a binder with specificity for one domain are recombined with light chain variable regions (V_{L}) from a series of binders with specificity for a second domain to identify non-cognate V_{L} partners that can be paired with an original V_{H} without disrupting the original specificity for that V_{H}. In this way, a single V_{L} segment (e.g., V_{L}1) can be combined with two different V_{H} domains (e.g., V_{H}1 and V_{H}2) to generate a bi-specific comprised of two binding “arms” (V_{H}1-V_{L}1 and V_{H}2-V_{L}1). Use of a single V_{L} segment reduces the complexity of the system and thereby simplifies and increases efficiency in cloning, expression, and purification processes used to generate the bi-specific (See, for example, U.S. Ser. No. 13/022,759 and US2010/0331527).

Alternatively, antigen-binding proteins that bind one domain and a second target, such as, but not limited to, for example, a second different anti-MBG antigen-binding protein, may be prepared in a bi-specific format using techniques described herein, or other techniques known to those skilled in the art. Immunoglobulin variable regions binding to MBG may be linked together with variable regions that bind to relevant sites on another cardiac glycoside such as ouabain, to confer dual-antigen specificity within a single binding molecule. Appropriately designed bi-specifics of this nature serve a dual function.

An exemplary bi-specific antigen-binding protein format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V4221 by EU) in the case of IgG1 antibodies; N44S, K52N, and V82I (IMGT; N384S, K392N, and V4221 by EU) in the case of IgG2 antibodies; and Q15R, N44S, K52N, V57M, R69K, E79Q, and V82I (by IMGT; Q355R, N384S, K392N, V397M, R409K, E419Q, and V4221 by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antigen-binding proteins can also be constructed using peptide/nucleic acid conjugation, e.g., wherein unnatural amino acids with orthogonal chemical reactivity are used to generate site-specific antigen-binding protein-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The invention provides therapeutic compositions comprising the anti-MBG antigen-binding proteins or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding protein may vary depending upon the age and the size of a subject to be administered, target disease, conditions, route of administration, and the like. When an antigen-binding protein of the present invention is used for treating a disease or disorder in an adult patient, or for preventing such a disease, it is advantageous to administer the antigen-binding protein of the present invention normally at a single dose of about 0.1 to about 100 mg/kg body weight, more preferably about 1 to about 60, about 5 to about 50, or about 10 to about 30 mg/kg body weight. Depending on the severity of the condition, the frequency and the duration of the treatment can be adjusted. In certain embodiments, the antigen-binding protein or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antigen-binding protein or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antigen-binding proteins of the present invention is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antigen-binding proteins contained in pharmaceutical compositions to target cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antigen-binding protein or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antigen-binding protein contained is generally about 5 to about 500 mg per dosage form in a unit dose; especially in the form of injection, it is preferred that the antigen-binding protein is contained in about 5 to about 100 mg and in about 10 to about 250 mg for the other dosage forms.

### Therapeutic Uses of the Antigen-Binding Proteins

The antigen-binding proteins of the present invention are useful for the treatment, and/or prevention of a disease or disorder or condition associated with MBG such as cardiovascular or renal disease and/or for ameliorating at least one symptom associated with such disease, disorder or condition. In one embodiment, an antigen-binding protein or antigen-binding fragment thereof the invention may be administered at a therapeutic dose to a patient with cardiovascular disease.

In certain embodiments, the antigen-binding proteins of the invention are useful to treat subjects suffering from cardiovascular disease, including volume expansion hypertension, myocardial fibrosis, uremic cardiomyopathy, heart failure, myocardial infarction, and pre-eclampsia. In some embodiments, the antigen-binding proteins of the invention are useful to treat subjects suffering from renal disease, including renal failure, and renal fibrosis. In one embodiment, the antigen-binding proteins of the invention are useful in reducing blood pressure in the subject.

One or more antigen-binding proteins of the present invention may be administered to relieve or prevent or decrease the severity of one or more of the symptoms or indications of the disease or disorder. The antigen-binding proteins may be used to ameliorate or reduce the severity of at least one symptom or indication of any MBG-associated disease or disorder including, but not limited to consisting of high blood pressure, atherosclerosis, hypertension, angina, shortness of breath, palpitations in the chest, weakness or dizziness, nausea, sweating, pressure or pain in the chest, arm or below the breastbone, irregular heartbeat, and death. In certain embodiments, the antigen-binding proteins of the present invention are useful to improve outcome and extend pregnancy in human subjects with pre-eclampsia.

It is also contemplated herein to use one or more antigen-binding proteins of the present invention prophylactically to subjects at risk for developing cardiovascular disease. The subjects at risk include, but are not limited to, an immunocompromised person, subjects of advanced age, pregnant women, and subjects with one or more risk factors including obesity, high blood cholesterol, smoking, excessive alcohol consumption, lack of exercise, and/or diabetes.

In a further embodiment of the invention the present antigen-binding proteins are used for the preparation of a pharmaceutical composition or medicament for treating patients suffering from a MBG-associated disease or disorder. In another embodiment of the invention, the present antigen-binding proteins are used as adjunct therapy with any other agent or any other therapy known to those skilled in the art useful for treating or ameliorating a MBG-associated disease or disorder such as cardiovascular or renal disease.

### Combination Therapies

Combination therapies may include an anti-MBG antigen-binding protein of the invention and any additional therapeutic agent that may be advantageously combined with an antigen-binding protein of the invention, or with a biologically active fragment thereof of the invention. The antigen-binding proteins of the present invention may be combined synergistically with one or more drugs or therapy used to treat any MBG-associated disease or disorder (e.g., cardiovascular disease). In some embodiments, the antigen-binding proteins of the invention may be combined with a second therapeutic agent to reduce the blood pressure in a subject, or to ameliorate one or more symptoms of cardiovascular disease.

The antigen-binding proteins of the present invention may be used in combination with an anti-hypertensive drug (e.g., an angiotensin-converting enzyme inhibitor, an angiotensin receptor blocker, a diuretic, a calcium channel blocker, an alpha-adrenoceptor blocker, an endothelin-1 receptor blocker, an organic nitrate, and a protein kinase C inhibitor), a statin, aspirin, a different antigen-binding protein to MBG, an inhibitor of ouabain or another cardiac glycoside, a dietary supplement such as anti-oxidants or any other therapy to treat cardiovascular disease.

As used herein, the term “in combination with” means that additional therapeutically active component(s) may be administered prior to, concurrent with, or after the administration of the anti-MBG antigen-binding protein of the present invention. The term “in combination with” also includes sequential or concomitant administration of an anti-MBG antigen-binding protein and a second therapeutic agent.

The additional therapeutically active component(s) may be administered to a subject prior to administration of an anti-MBG antigen-binding protein of the present invention. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-MBG antigen-binding protein of the present invention. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-MBG antigen-binding protein of the present invention. “Concurrent” administration, for purposes of the present invention, includes, e.g., administration of an anti-MBG antigen-binding protein and an additional therapeutically active component to a subject in a single dosage form, or in separate dosage forms administered to the subject within about 30 minutes or less of each other. If administered in separate dosage forms, each dosage form may be administered via the same route (e.g., both the anti-MBG antigen-binding protein and the additional therapeutically active component may be administered intravenously, etc.); alternatively, each dosage form may be administered via a different route (e.g., the anti-MBG antigen-binding protein may be administered intravenously, and the additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of an anti-MBG antigen-binding protein “prior to”, “concurrent with,” or “after” (as those terms are defined herein above) administration of an additional therapeutically active component is considered administration of an anti-MBG antigen-binding protein “in combination with” an additional therapeutically active component.

The present invention includes pharmaceutical compositions in which an anti-MBG antigen-binding protein of the present invention is co-formulated with one or more of the additional therapeutically active component(s) as described elsewhere herein.

### Administration Regimens

According to certain embodiments, a single dose of an anti-MBG antigen-binding protein of the invention (or a pharmaceutical composition comprising a combination of an anti-MBG antigen-binding protein and any of the additional therapeutically active agents mentioned herein) may be administered to a subject in need thereof. According to certain embodiments of the present invention, multiple doses of an anti-MBG antigen-binding protein (or a pharmaceutical composition comprising a combination of an anti-MBG antigen-binding protein and any of the additional therapeutically active agents mentioned herein) may be administered to a subject over a defined time course. The methods according to this aspect of the invention comprise sequentially administering to a subject multiple doses of an anti-MBG antigen-binding protein of the invention. As used herein, “sequentially administering” means that each dose of anti-MBG antigen-binding protein is administered to the subject at a different point in time, e.g., on different days separated by a predetermined interval (e.g., hours, days, weeks or months). The present invention includes methods which comprise sequentially administering to the patient a single initial dose of an anti-MBG antigen-binding protein, followed by one or more secondary doses of the anti-MBG antigen-binding protein, and optionally followed by one or more tertiary doses of the anti-MBG antigen-binding protein.

The terms “initial dose,” “secondary doses,” and “tertiary doses,” refer to the temporal sequence of administration of the anti-MBG antigen-binding protein of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-MBG antigen-binding protein, but generally may differ from one another in terms of frequency of administration. In certain embodiments, however, the amount of anti-MBG antigen-binding protein contained in the initial, secondary and/or tertiary doses varies from one another (e.g., adjusted up or down as appropriate) during the course of treatment. In certain embodiments, one or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 48 hours (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) after the immediately preceding dose. The phrase “the immediately preceding dose,” as used herein, means, in a sequence of multiple administrations, the dose of anti-MBG antigen-binding protein which is administered to a patient prior to the administration of the very next dose in the sequence with no intervening doses.

The methods according to this aspect of the invention may comprise administering to a patient any number of secondary and/or tertiary doses of an anti-MBG antigen-binding protein. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

### Diagnostic Uses of the Antigen-Binding Proteins

The anti-MBG antigen-binding proteins of the present invention may be used to detect and/or measure MBG in a sample, e.g., for diagnostic purposes. Some embodiments contemplate the use of one or more antigen-binding proteins of the present invention in assays to detect a disease or disorder such as cardiovascular disease (e.g., hypertension, cardiomyopathy, and pre-eclampsia). Exemplary diagnostic assays for MBG may comprise, e.g., contacting a sample, obtained from a subject, with an anti-MBG antigen-binding protein of the invention, wherein the anti-MBG antigen-binding protein is labeled with a detectable label or reporter molecule or used as a capture ligand to selectively isolate MBG from subject samples. Alternatively, an unlabeled anti-MBG antigen-binding protein can be used in diagnostic applications in combination with a secondary antibody which is itself detectably labeled. The detectable label or reporter molecule can be a radioisotope, such as ³H, ¹⁴O, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure MBG in a sample include enzyme-linked immunosorbent assay (ELISA), solid phase, fluroimmunoassay, radioimmunoassay (RIA), and fluorescence-activated cell sorting (FACS).

Samples that can be used in MBG diagnostic assays according to the present invention include any tissue or fluid sample obtainable from a subject, which contains detectable quantities of MBG, under normal or pathological conditions (e.g., plasma, serum and urine). Generally, levels of MBG in a particular sample obtained from a healthy patient (e.g., a patient not afflicted with a disease associated with MBG) will be measured to initially establish a baseline, or standard, level of MBG. This baseline level of MBG can then be compared against the levels of MBG measured in samples obtained from individuals suspected of having a MBG-associated condition, or symptoms associated with such condition.

The antigen-binding proteins specific for MBG may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

### Examples

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antigen-Binding Proteins to MBG

Human antigen-binding proteins to marinobufagenin were generated in a genetically modified mouse whose genome comprised (i) an immunoglobulin heavy chain allele that contained an insertion of 40 unrearranged human Vκ and 5 Jκ gene segments so that said gene segments were operably linked to endogenous heavy chain constant regions, and (ii) an immunoglobulin light chain allele that contained an insertion of 40 unrearranged human Vκ and 5 Jκ gene segments so that said segments were operably linked to an endogenous light chain constant region (referred to as “KOH mice”, see US Patent Application Publication US2012/0096572, herein incorporated in its entirety). Antibodies produced by such mice comprise two light chain variable domain portions rather a traditional pairing of a heavy chain variable region and a light chain variable region. The mice were immunized with MBG conjugated to BSA followed by one or more booster dose(s).

The immune response was monitored by a MBG-specific immunoassay. Anti-MBG antigen-binding proteins were isolated directly from antigen-positive mouse B cells without fusion to myeloma cells, as described in U.S. Pat. No. 7,582,298, herein specifically incorporated by reference in its entirety. Using this method, several fully human anti-MBG antigen-binding proteins (i.e., antigen-binding proteins possessing human variable domains and human constant domains) were obtained; exemplary antigen-binding proteins generated in this manner were designated as H4H14357P, H4H14362P, H4H14368P, H4H14371 P, H4H14372P, H4H14373P, H4H14401 P, H4H14407P, H4H14416P, H4H14417P, and H4H14389P.

The biological properties of the exemplary antigen-binding proteins generated in accordance with the methods of this Example are described in detail in the Examples set forth below.

### Example 2: Variable Region Amino Acid and Nucleotide Sequences

Table 1 sets forth the amino acid sequence identifiers of the first and second variable regions (VR1 and VR2) and CDRs of selected anti-MBG antigen-binding proteins of the invention.

TABLE 1
Amino Acid Sequence Identifiers

| Antigen- | | | | | | | | |
|---|---|---|---|---|---|---|---|---|
| binding | | | | | | | | |
| Protein | SEQ ID NOs: | | | | | | | |
| Designation | VR1 | CDR1 | CDR2 | CDR3 | VR2 | CDR4 | CDR5 | CDR6 |
| H4H14357P | 2 | 4 | 6 | 8 | 10 | 12 | 14 | 16 |
| H4H14362P | 18 | 20 | 22 | 24 | 26 | 28 | 30 | 32 |
| H4H14368P | 34 | 36 | 38 | 40 | 42 | 44 | 46 | 48 |
| H4H14371P | 50 | 52 | 54 | 56 | 58 | 60 | 62 | 64 |
| H4H14372P | 66 | 68 | 70 | 72 | 74 | 76 | 78 | 80 |
| H4H14373P | 82 | 84 | 86 | 88 | 90 | 92 | 94 | 96 |
| H4H14401P | 98 | 100 | 102 | 104 | 106 | 108 | 110 | 112 |
| H4H14407P | 114 | 116 | 118 | 120 | 122 | 124 | 126 | 128 |
| H4H14416P | 130 | 132 | 134 | 136 | 138 | 140 | 142 | 144 |
| H4H14417P | 146 | 148 | 150 | 152 | 154 | 156 | 158 | 160 |
| H4H14389P | 162 | 164 | 166 | 168 | 170 | 172 | 174 | 176 |

The corresponding nucleic acid sequence identifiers are set forth in Table 2.

TABLE 2
Nucleic Acid Sequence Identifiers

| Antigen- | | | | | | | | |
|---|---|---|---|---|---|---|---|---|
| binding | | | | | | | | |
| Protein | SEQ ID NOs: | | | | | | | |
| Designation | VR1 | CDR1 | CDR2 | CDR3 | VR2 | CDR4 | CDR5 | CDR6 |
| H4H14357P | 1 | 3 | 5 | 7 | 9 | 11 | 13 | 15 |
| H4H14362P | 17 | 19 | 21 | 23 | 25 | 27 | 29 | 31 |
| H4H14368P | 33 | 35 | 37 | 39 | 41 | 43 | 45 | 47 |
| H4H14371P | 49 | 51 | 53 | 55 | 57 | 59 | 61 | 63 |
| H4H14372P | 65 | 67 | 69 | 71 | 73 | 75 | 77 | 79 |
| H4H14373P | 81 | 83 | 85 | 87 | 89 | 91 | 93 | 95 |
| H4H14401P | 97 | 99 | 101 | 103 | 105 | 107 | 109 | 111 |
| H4H14407P | 113 | 115 | 117 | 119 | 121 | 123 | 125 | 127 |
| H4H14416P | 129 | 131 | 133 | 135 | 137 | 139 | 141 | 143 |
| H4H14417P | 145 | 147 | 149 | 151 | 153 | 155 | 157 | 159 |
| H4H14389P | 161 | 163 | 165 | 167 | 169 | 171 | 173 | 175 |

The antigen-binding proteins of Table 1 are fully human antigen-binding proteins comprising a human IgG4 Fc. As will be appreciated by a person of ordinary skill in the art, an antigen-binding protein having a particular Fc isotype can be converted to an antigen-binding protein with a different Fc isotype (e.g., an antigen-binding protein with a mouse IgG1 Fc can be converted to an antigen-binding protein with a human IgG4, etc.), but in any event, the variable domains (including the CDRs)—which are indicated by the numerical identifiers shown in Table 1—will remain the same, and the binding properties to antigen are expected to be identical or substantially similar regardless of the nature of the Fc domain. According to certain embodiments, the Fc region of the antigen-binding proteins of the present invention comprises amino acid sequences of SEQ ID NOs: 183, 184, 185, 186 or 187.

### Control Construct Used in the Following Examples

The following control construct (anti-MBG antibody) was included in the experiments disclosed herein, for comparative purposes: “Comparator 1,” a monoclonal antibody with mouse IgG4 against MBG having V_{H}/V_{L} sequences of antibody “3E9” according to Fedorova et al 2008, J. Hypertens. 26: 2414-25.

### Example 3: Binding to MBG as Determined by Isothermal Titration Calorimetry

Affinities for antigen-binding proteins binding to MBG were determined using isothermal titration calorimetric (ITC) methods with MicroCal™ Auto-iTC₂₀₀ (GE Healthcare). All experiments were run in titration buffer (degassed PBS; 0.01M Na₂HPO₄/NaH₂PO₄, pH 7.4 and 0.15 M NaCl, containing 1.6% (v/v) DMSO). Stock solutions of anti-MBG antigen-binding proteins were dialyzed against or desalted in degassed PBS buffer and serially diluted with PBS buffer to achieve final concentrations and 1.6% DMSO (v/v) was added to match the titration buffer. Absorbance of anti-MBG antigen-binding proteins was measured at 280 nm on a Nanodrop UV spectrophotometer, with extinction coefficients derived from individual anti-MBG antigen-binding protein sequences. Final concentration of MBG (Catalog # M0093; NIH) in PBS (75 μM or 150 μM) was readjusted for final DMSO concentration of 1.6% to match the titration buffer formulation. Anti-MBG antigen-binding proteins and MBG solutions were centrifuged (10×g, 10 minutes at RT) prior to running ITC experiments.

A typical ITC experiment involved injecting a set number of fixed aliquots of ligand from a syringe, into the ITC cell containing binding partner. From each injection, the power needed to maintain a constant temperature in both reaction cell and the reference cell, namely the energy change (qᵢ), was measured and plotted as an isotherm. Non-linear least squares fit of the binding isotherm provides binding constants (K_{A} and K_{D}=1/K_{A}), enthalpy (ΔH) and binding stoichiometric (N) values. Free energy (ΔG) and entropy (ΔS) of the interaction are derived from the K_{A} values, using the equations;

ΔG=−RT(ln K_{A})(R: gas constant,T: absolute temperature)

ΔS=(ΔH−ΔG)/T

All MicroCal™ Auto-iTC₂₀₀ based experiments were performed in the automated workflow mode, including all sample introductions, titration, and cleaning steps. Titrations were performed with 20-fold excess of MBG injections (2 μL in 4 seconds) from the syringe, into the ITC cell containing anti-MBG antigen-binding proteins (4.6 μM-11 μM, Table 3). Reference power was set at 6 μcal/second, and interval between injections was 180 seconds with the cell maintained at 25° C. and constantly stirred at 750 rpm. Both the cell and syringe were extensively washed (sequentially with detergent, water and titration buffer) between successive experiments. ITC-customized Origins 7.0 software was used to analyze data and the resulting isotherms were fitted using non-linear least squares method with one-site model conditions. Binding parameters (N, K_{A}, K_{D}, ΔH, ΔG and TΔS) was determined for 14 anti-MBG antigen-binding proteins and are recorded in Table 3.

TABLE 3
Thermodynamic binding parameters of MBG binding to anti-MBG antigen-binding proteins at 25° C.

| | Antigen- | | | | | | |
|---|---|---|---|---|---|---|---|
| Antigen- | binding | MBG | | | | | |
| binding | protein conc. | Conc. in | | | | | |
| protein | in cell (μM) | syringe (μM) | N | K_{A} (1/M) | K_{D} (M) | ΔH (cal/mol) | (−)TΔS (cal/mol) |
| H4H14357P | 5.6 | 75 | 1.97 | 2.87E+07 | 3.48E−08 | −9135 | −1037 |
| H4H14362P | 5.9 | 75 | 1.59 | 5.60E+07 | 1.79E−08 | −14460 | 3904 |
| H4H14368P | 5.7 | 75 | 1.95 | 1.90E+08 | 5.26E−09 | −14130 | 2837 |
| H4H14389P | 5.3 | 75 | 1.89 | 2.44E+07 | 4.10E−08 | −17340 | 7271 |
| H4H14417P | 5.5 | 75 | 1.62 | 3.06E+07 | 3.27E−08 | −14160 | 3934 |
| H4H14416P | 5.8 | 75 | 1.9 | 2.11E+07 | 4.74E−08 | −12690 | 2697 |
| H4H14401P | 5.3 | 75 | 1.81 | 1.59E+08 | 6.29E−09 | −18850 | 7659 |
| H4H14407P | 10.7 | 150 | 1.85 | 1.27E+06 | 7.87E−07 | −9984 | 1660 |
| H4H14371P | 11 | 150 | 1.72 | 2.08E+07 | 4.81E−08 | −11820 | 1842 |
| H4H14372P | 11 | 150 | 1.63 | 1.51E+07 | 6.62E−08 | −11480 | 1690 |
| H4H14373P | 11 | 150 | 2.03 | 2.18E+07 | 4.59E−08 | −12690 | 2682 |
| Comparator 1 | 10 | 178.6 | 2.16 | 4.93E+06 | 2.03E−07 | −7557 | −1570 |

All 11 anti-MBG antigen-binding proteins of the invention bound to MBG with K_{D} values ranging from 5.26 nM to 787 nM. The isotype control did not show any binding (data not shown in the table).

### Example 4: Binding to MBG as Determined by Surface Plasmon Resonance

Surface plasmon resonance (SPR) experiments were performed on a Biacore 2000 instrument using a dextran-coated (CM5) chip at 25° C. The running buffer was filtered PBS (8.1 mM Na2HPO4, 1.9 mM NaH2PO4, 2.7 mM KCl, 137 mM NaCl, 0.1% v/v DMSO, adjusted to pH7.4). A capture sensor surface was prepared by covalently immobilizing recombinant Protein A (Pierce, Rockford, Ill.) to the chip surface using (1 Ethyl-3-[3-dimethylaminopropyl]carbodiimide hydrochloride)/N-hydroxysuccinimide (EDC/NHS) coupling chemistry. Following surface activation, Protein A in coupling buffer (0.1 M acetate buffer, pH 4.5) was injected over the activated chip surface until a resonance unit (RU) signal of about 2000 RU was reached. The activated coupled chip surfaces were then washed and treated with 10 mM glycine-HCl, pH 1.5, to remove uncoupled residual Protein A.

Anti-MBG antigen-binding proteins were diluted into the running buffer and captured on the coupled Protein A chip surface. Following the capture step, a range of concentration of MBG (270.0 μM to 13.7 nM) were individually injected over different anti-MBG antigen-binding protein-captured surfaces. For all ligands, the association rate constant (kₐ) was determined from data obtained at multiple test ligand concentrations. The dissociation rate constant (k_{d}), which is independent of test ligand concentration, was determined from the change in anti-MBG bound test ligand RU over time (˜5 minutes) for MBG ligands. Specific Biacore kinetic sensorgrams were obtained by a double referencing procedure as described by Myszka et al 1999, J. Mol. Recognit. 12: 279-84. The data were then processed and kinetic analyses performed using Scrubber software (version 2.0, BioLogic Software). The equilibrium dissociation constant (K_{D}) was calculated from the ratio of the dissociation rate constant divided by the association rate constant (K_{D}=k_{d}/kₐ).

The equilibrium dissociation constant (KD) between anti-MBG antigen-binding proteins and MBG was measured using SPR-Biacore technology. Kinetic binding data were generated using an amine-coupled Protein A surface and subsequent anti-MBG monoclonal capture at high density. KD values for the interaction between four anti-MBG antigen-binding proteins and MBG ranged from 4.4 nM to 1.9 μM (Table 4).

TABLE 4
Binding kinetics of MBG binding to
anti-MBG antigen-binding proteins

| | | | KD (M) | t½ |
|---|---|---|---|---|
| Antibody | ka (M−1s−1) | kd (s−1) | SPR | (seconds) |
| Comparator 1 | 1.03E+06 | 5.85E−02 | 5.68E−08 | 12 |
| H4H14357P | 4.82E+06 | 4.58E−02 | 9.50E−09 | 15 |
| H4H14362P | 6.40E+05 | 7.43E−03 | 1.16E−08 | 93 |
| H4H14368P | IC | IC | IC | IC |
| H4H14371P | 1.35E+06 | 5.98E−03 | 4.43E−09 | 116 |
| H4H14372P | 1.19E+06 | 7.11E−03 | 5.97E−09 | 97 |
| H4H14373P | 8.50E+05 | 7.41E−03 | 8.72E−09 | 94 |
| H4H14389P | NB | NB | NB | NB |
| H4H14401P | 1.01E+06 | 4.46E−03 | 4.42E−09 | 156 |
| H4H14407P | 1.04E+05 | 2.02E−01 | 1.94E−06 | 3 |
| H4H14416P | 2.42E+06 | 8.10E−02 | 3.35E−08 | 9 |
| H4H14417P | IC | IC | IC | IC |

NB: no binding;
IC: inconclusive

The response data for the binding of MBG to different anti-MBG antigen-binding protein captured surface densities of MBG were globally fitted simultaneously to a simple 1:1 interaction model, constraining the kinetic rate constants to a single value. The KD for highest affinity for anti-MBG protein binding to MBG was approximately 13-fold lower (i.e. the binding affinity was ˜13-fold tighter) than that for control.

### Example 5: Isoelectric Point Determination

The isoelectric point (pI) determination and charge variant profiling of anti-MBG antigen-binding proteins was performed by imaged capillary isoelectric focusing using a iCE3 analyzer equipped with Alcott 720 NV autosampler (ProteinSimple, San Jose, Calif.). The catholyte tank was filled with 0.1 M sodium hydroxide in 0.1% methyl cellulose and the anolyte tank was filled with are 0.08 M phosphoric acid in 0.1% methyl cellulose. Anti-MBG antigen-binding proteins were focused at 1500 V for 1 min and then at 3000 V for 7 min. Focused protein bands were detected by UV absorbance at 280 nm. The electropherograms were exported and processed in Empower 3 software (Waters Corp., Milford, Mass., USA). Samples applied to the iCE3 analyzer contained a basic pI marker, an acidic pI marker, pharmalyte 3-10, 2M urea and 0.5 mg/mL of protein.

To look at post-translational modifications (e.g. glycosylation, deamidation etc.) that lead to charge heterogeneity, imaged capillary isoelectric focusing (iCIEF) was used to determine pI of all charge variant species present in selected anti-MBG antigen-binding proteins. On average 5-8 charge species were resolved for each one. The range of pI values for all the observed species was between 7.1-7.9 for the anti-MBG antigen-binding proteins. The charge species with the highest peak area was defined as ‘main peak’ and the pI of this peak was reported as the pI of the molecule. The pIs of H4H14357P, H4H14371 P, H4H14401 P and isotype control were 7.7, 7.6, 7.6 and 5.9 respectively.

### Example 6: Thermal Stability of Anti-MBG Antigen-Binding Proteins

The unfolding or denaturation (Tₘ) temperatures for anti-MBG antigen-binding proteins were measured by differential scanning calorimetry (DSC). Anti-MBG proteins were diluted to 1 mg/mL in reference buffer (10 mM Histidine, pH 5.5). Diluted samples and buffer reference were degassed and equilibrated for 5 min at 10° C. Following degassing, samples were subjected to a temperature ramp at a scan rate of 90° C./hour to a maximum of 105° C. on a MicroCal VP-DSC Capillary Cell MicroCalorimeter (Malvern Instruments, Westborough, Mass., USA). Baseline correction and concentration normalization was applied to all data. Origin 7 (OriginLab, Northampton, Mass.) software was used to fit data to a No 2-State model (two transitions) to determine T, values.

Three transitions were most commonly observed during DSC analysis of anti-MBG antigen-binding proteins H4H14357P, H4H14401P and H4H14371P corresponding to the unfolding of CH2, Fab and CH3 domains respectively. Depending on the molecular structure, not all the transitions were resolved under experimental conditions used. Data fitting models were used to differentiate and resolve the transitions. Only one peak was observed in the thermograms of samples in this study. Fitting to a non 2-state model provided the best fit for the observed thermograms. Two thermal transitions for each anti-MBG antigen-binding protein were determined from the model.

Generally, higher Tm values are indicative of better stability due to an increased resistance to unfolding under thermal stress. In addition to the inherent stability of the antibody, formulation excipients and other solution conditions may have a stabilizing effect by increasing the temperature of thermal transition. The anti-MBG antigen-binding proteins in this study exhibited Tm values >60° C. that would indicate lower risk of instability under higher temperatures.

### Example 7: Membrane Potential Assay with HEK293/hnAChR α3/β4 Cells

An in-vitro cell-based assay was developed to assess the regulation of Na+/K+ ATPase by MBG using HEK293 cells. HEK293 has Na+/K+ ATPase activity and contains the α1 isoform (Kochskämper et al. 1997, Biochim. Biophys. Acta 1325: 197-208). MBG is thought to preferentially inhibit the α1 isoform containing Na+/K+ ATPase (Fedorova and Bagrov 1997, Am. J. Hypertens. 10: 929-935; Fedorova et al. 2000, Circulation 102: 3009-3014). HEK293 cells expressing human neuronal nicotinic acetylcholine receptors (nAChR) subunits α3 and β4 (Accession # M37981 and #NM_000750), HEK293/hnAChR α3/β4 (Millipore) were used. The nAchR heterodimer is a ligand-gated ion channel that mediates influx of Na+ upon ligand binding (Corringer et al. 2000, Annu. Rev. Pharmacol. Toxicol. 40: 431-458). Epibatidine, a potent agonist of nAchR, was used to activate the channel, causing an influx on Na⁺ into the cells, thereby depolarizing cell membranes and activating Na⁺/K⁺ ATPase. MBG was added to inhibit the repolarization of the cell through its effects on Na⁺/K⁺ ATPase. Changes in the membrane potential of the cells were monitored using a fluorescent dye whose intensity increases when the cells are depolarized and decreases as the cells are repolarized.

For the bioassay, cells were detached with Enzyme Free Cell Dissociation Buffer (Millipore) seeded into 96-well assay plates at 50,000 cells/well in Opti-MEM™ assay buffer supplemented with 0.1% FBS, penicillin/streptomycin and L-glutamine (known from this point forward as Opti-MEM™), and then incubated at 37° C. in 5% CO₂ overnight. The next day, membrane potential dye (Molecular Devices) dissolved in loading buffer was added to cells. After addition of the membrane potential dye, the cells were incubated at 37° C. in 5% CO₂ for 30 minutes, followed by incubation at 25° C. for 30 minutes. The plate was then placed into the FLIPR^{Tetra}® (Molecular Devices) and kinetic readings were collected at 25° C. All dilutions were prepared in Opti-MEM™ assay buffer. In order to elicit membrane depolarization, Epibatidine was serially diluted 1:3 from 1000 nM to 17 pM or from 300 nM to 412 pM (both including a control sample containing no Epibatidine) and added to cells. To measure the ability of MBG to inhibit membrane repolarization through inhibition of Na⁺/K⁺ ATPase, Epibatidine, prepared at a constant concentration of 100 nM, was added to cells and after 100 or 200 seconds, serially diluted MBG at 1:3 from 5000 nM to 85 pM or to 726 pM (including a control sample containing no MBG) was added to cells. To measure the blocking of MBG inhibition of membrane repolarization, anti-MBG antigen-binding proteins serially diluted at 1:3 from 1000 nM to 12.3 nM or from 1800 nM to 22.2 nM (both including a control sample containing no antigen-binding protein) were incubated with 400 nM or 700 nM of MBG for 30 minutes at 25° C. Epibatidine, prepared at a constant concentration of 100 nM, was added to cells and after 100 or 200 seconds the pre-incubated mixture of anti-MBG antigen-binding proteins and MBG was added to cells.

Blocking of MBG activity by anti-MBG antigen-binding proteins without pre-incubation of MBG was also tested. In this case 700 nM MBG and 100 nM Epibatadine were initially added to the cell simultaneously and serially diluted anti-MBG antigen-binding proteins (diluted at 1:3 from 1800 nM to 22 nM, including a sample containing no antigen-binding protein) were added to the cells 200 seconds later.

For the kinetic reading using the FLIPR^{Tetra}®, the fluorescence of each well was measured for the first 100 or 200 seconds after the addition of the Epibatidine. At this point, the MBG and/or the anti-MBG antigen-binding proteins were added to the cells, with fluorescence of each well then measured until the conclusion of the experiment, 30 or 35 minutes after the second addition to the cells. Fluorescence readings, measured in relative fluorescence units (RFUs) were collected with an excitation filter of 510-545 nm and an emission filter of 565-625 nm.

To analyze the data for each well, the RFU value from the end of the experiment was subtracted from the RFU value immediately before the second addition was made to the cells to generate a ΔRFU value. The ΔRFU value is a measure of the change of membrane potential such that the ΔRFU value is high when a maximal membrane repolarization follows a membrane depolarization event elicited by Epibatidine without the presence of MBG. For a fixed concentration of Epibatidine, the ΔRFU value decreases with increasing concentration of MBG as MBG inhibits membrane repolarization. In contrast, with fixed concentrations of Epibatidine and MBG, the ΔRFU value increases with increasing concentration of anti-MBG antigen-binding proteins as they inhibit MBG function, causing an increasing amount of membrane repolarization. These ΔRFU values were analyzed as a function of concentration using nonlinear regression (4-parameter logistics) with Prism 6 software (GraphPad) to obtain EC₅₀ and IC₅₀ values.

TABLE 5
Blocking of 400 nM MBG inhibition of membrane repolarization
of HEK293/hnAChRα3/β4 cells following membrane
depolarization by 100 nM Epibatidine

| EC₅₀ [M] of Epibatidine | 5.9E−09 |
|---|---|
| IC₅₀ [M] of MBG | 7.0E−07 |
| (@ 100 nM Epibatidine) | |
| Constant concentrations | 400 nM MBG and 100 nM Epibatidine |
| for inhibition by antigen- | |
| binding proteins | |
| Antigen-binding Protein | EC₅₀ [M] |
| H4H14357P | 8.2E−08 |
| H4H14362P | >3.0E−07 |
| H4H14368P | 9.5E−08 |
| H4H14371P | 7.5E−08 |
| H4H14372P | 1.1E−07 |
| H4H14373P | 1.4E−07 |
| H4H14401P | 1.0E−07 |
| H4H14407P | >3.0E−07 |
| H4H14416P | >3.0E−07 |
| H4H14417P | 1.5E−07 |
| Comparator 1 | 1.0E−07 |
| Isotype control antibody | No inhibition |

As shown in Table 5, 7 out of 10 of the anti-MBG antigen-binding proteins of the invention exhibited inhibition of 400 nM of MBG inhibition of membrane repolarization of HEK293/hnAChRα3/β4 cells following membrane depolarization by 100 nM Epibatidine, with EC₅₀ values ranging from 75 nM to 150 nM. Three anti-MBG antigen-binding proteins of the invention showed EC₅₀ values that are greater than 300 nM. The isotype control antibody did not demonstrate any measurable inhibition in this assay. Epibatidine showed increased membrane depolarization with an EC₅₀ of 5.9 nM and MBG showed inhibition of membrane repolarization elicited by 100 nM Epibatidine with an IC₅₀ of 700 nM.

MBG inhibition by the anti-MBG antigen-binding proteins when added to the cells 200 seconds following the simultaneous addition of 700 nM MBG and 100 nM Epibatadine to the cells was also tested.

TABLE 6
Blocking of 700 nM MBG inhibition of membrane repolarization
of HEK293/hnAChRα3/β4 cells following membrane depolarization
by 100 nM Epibatidine with or without pre-incubation with
MBG with antigen-binding proteins

| EC₅₀ [M] of Epibatidine | 1.3E−09 | 2.3E−09 |
|---|---|---|
| MBG Addition Condition | MBG added after Epibatidine | MBG added with Epibatidine |
| IC₅₀ [M] of MBG | 6.6E−07 | 4.5E−07 |
| (@ 100 nM Epibatidine) | | |
| Constant concentrations for | 700 nM MBG and 100 nM Epibatidine | |
| inhibition by antigen-binding | | |
| proteins | | |
| Antigen-binding protein | Antigen-binding protein pre- | Antigen-binding protein |
| Addition Condition | incubated with MBG | added after MBG |
| Antibody | EC₅₀ [M] | EC₅₀ [M] |
| H4H14371P | 7.5E−08 | 1.2E−07 |
| H4H14401P | 8.5E−08 | 1.2E−07 |
| H4H14357P | 1.2E−07 | 1.7E−07 |
| Comparator 1 | 2.2E−07 | 2.4E−07 |
| Isotype control antibody 2 | No inhibition | No inhibition |

As shown in Table 6, all 3 anti-MBG antigen-binding proteins tested showed complete inhibition of 700 nM MBG when added to cells following MBG addition with EC₅₀ values of 120-170 nM similar to the EC₅₀ values achieved by the antigen-binding proteins pre-incubated with MBG of 75-120 nM. The isotype control antibody did not demonstrate any measurable inhibition in this assay both with and without MBG pre-incubation. Epibatidine showed increased membrane depolarization with EC₅₀ values of 1.3 nM and 2.3 nM and MBG showed inhibition of membrane repolarization elicited by 100 nM Epibatidine with 10₅₀ values of 446 nM for MBG added simultaneously with Epibatidine and 662 nM for MBG added 200 seconds after Epibatidine.

### Example 8: In Vivo Efficacy of Anti-MBG Antigen-Binding Proteins

The objective of this study was to assess the efficacy of anti-MBG antigen-binding proteins to alter hemodynamic and renal function in the Dahl/Salt Sensitive rat. Male Dahl/Salt Sensitive rats (SS/JrHsdMcwiCrl) (n=39) aged ˜8 weeks were implanted with PA-C40 telemeters (DSI, St. Paul, Minn.) and allowed to recover for 7 days, prior to being assigned to group (Groups 1-7) (Table 7). Animals were individually housed under standard conditions (Temperatures of 64° F. to 84° F. (18° C. to 29° C.); relative humidity of 30% to 70%) and a 12-hour light/12-hour dark cycle was maintained. Food and water were provided ad libitum. Two different diets were used: 1) control diet, AIN-76A (#5800-B) provided to Group 1 and 2) high salt diet, AIN-76A with 8% NaCl (STRC) provide to Groups 2-7.

The test proteins were administered to the appropriate animals by intraperitoneal injection on Days 1, 7, 21, and 22 and doses were administered by subcutaneous injection on Day 35. The dose volume for each animal was based on the most recent body weight measurement.

Blood was collected from a jugular vein approximately 6 hr post-dose. Urine was collected over 24 hours, beginning approximately 1.5 hr after dosing was completed. After collection, samples were transferred to the appropriate laboratory for processing.

Heart rate, systolic pressure, diastolic pressure and mean pressure, heart rate, and activity were collected for 10 seconds every minute. Data were recorded for at least 24 hours during Week −3 and Week −1. On days of dosing, telemetry recordings were initiated at least 2 hours prior to dosing and continued for at least 48 hours after dosing.

TABLE 7
Experimental design

| | | | | | | Number |
|---|---|---|---|---|---|---|
| | | | | | | of |
| Group | | Test or | Dose | | Dose | Animals |
| No. | Diet | Control | Level (mg/kg) | Study Days | Volume (mL/kg) | Males |
| 1 | AIN-76A | Saline | 0 | 1, 7, 21, 22 | 10 | 3 |
| | (#5800-B) | Saline | 0 | 35 | 5 | |
| 2 | AIN-76A with | Saline | 0 | 1, 7, 21, 22 | 10 | 1 |
| | 8% NaCl | Saline | 0 | 35 | 5 | |
| 3 | (5TRC) | Comparator 1 | 5 | 1 | 10 | 6 |
| | | | 15 | 7 | | |
| | | | 25 | 21, 22 | | |
| | | | 100 | 35 | 5 | |
| 4 | | Isotype | 1.8 | 1 | 10 | 6 |
| | | Control | 5.3 | 7 | | |
| | | | 8.8 | 21, 22 | | |
| | | | 35.2 | 35 | 5 | |
| 5 | | H4H14371P | 4.9 | 1 | 10 | 6 |
| | | | 14.8 | 7 | | |
| | | | 24.6 | 21, 22 | | |
| | | | 98.35 | 35 | 5 | |
| 6 | | H4H14357P | 4.8 | 1 | 10 | 6 |
| | | | 14.5 | 7 | | |
| | | | 24.1 | 21, 22 | | |
| | | | 96.45 | 35 | 5 | |
| 7 | | H4H14401P | 4.8 | 1 | 10 | 6 |
| | | | 14.5 | 7 | | |
| | | | 24.1 | 21, 22 | | |
| | | | 96.45 | 35 | 5 | |

The high salt diet caused an increase in blood pressure as expected. At Week −3, systolic blood pressure was similar in high salt animals and controls. Systolic blood pressure was higher in salt-fed animals in Week −2 compared to the controls (˜20 mmHg higher in high salt animals). By Week −1, the difference between high salt fed animals and control animals exceeded 40 mmHg. By Day 35, systolic pressure in the control animals remained fairly stable at ˜140 mmHg while systolic pressure was ˜200 mmHg or higher in the animals that had been on the high salt diet. Heart rates were similar in both high salt and control animals, ranging between 300 and 450 beats per minute. The rats demonstrated an expected diurnal rhythm with higher blood pressure and heart rate during the nighttime period and lower heart rate and blood pressure during daytime periods.

On Day 21, all of the test proteins, including the isotype control, caused an approximate 40-70 mmHg reduction in systolic pressure which peaked ˜20-30 minutes post dose. Blood pressure recovered slowly to control levels by ˜2 to 3 hours post dose. The reduction in blood pressure was followed by a compensatory increase in heart rate (˜100 bpm compared to baseline). Heart rate had returned to control levels by ˜6 hours post dose.

On Day 22, blood pressure was reduced in animals treated with H4H14371P, H4H14357P, and H4H14401P. Animals treated with H4H14371P and H4H14401P had systolic pressure reduced ˜20 mmHg from baseline while those animals treated with H4H14357P had systolic pressure reduced by ˜40 mmHg when compared to baseline. The reductions in systolic pressure peaked between 5 and 30 minutes post dose and pressure returned to baseline levels ˜2.5 hours post dose. Heart rate did not appear to be affected with the repeat dose on Day 22. Comparator 1 and control did not elicit a change in blood pressure or heart rate on Day 22.

On Day 35, the 100 mg/kg dose of Comparator 1 caused an approximate 55 mmHg reduction in systolic pressure which peaked ˜30-35 minutes post dose. H4H14357P at 96.45 mg/kg caused a similar reduction in systolic pressure (˜60 mmHg) which peaked 25 minutes post dose. H4H14371P at 98.35 mg/kg resulted in an approximate 30 mmHg reduction in systolic pressure which peaked 25 minutes post dose. Control and H4H14401P at 35.2 and 96.45 mg/kg, respectively, caused an approximate 20 mmHg reduction in systolic pressure. Blood pressure in all animals returned to baseline levels by approximately 90 minutes post dose. Heart rate was increased ˜20-60 bpm for 2-2.5 hours post dose.

There was no change in blood pressure or heart rate with the 10 mg/kg dose of Comparator 1 at 10 mg/kg on Day 28.

On Days 1 and 7, none of the doses altered blood pressure or heart rate.

Clinical chemistry changes associated with the salt-induced hypertension and secondary nephropathy included decreased potassium and increased cholesterol and triglycerides. Some individual animals also had increased creatinine and blood urea nitrogen. These changes were expected characteristics of the model. Urine chemistry changes associated with the salt-induced hypertension and secondary nephropathy included increased urine volume and proteinuria and decreased creatinine and urea nitrogen.

There were no apparent treatment-related changes in body weight or food consumption. There were no apparent treatment-related changes in clinical chemistry or urine chemistry parameters.

Nine animals were either euthanized early due to declining clinical condition (unscheduled euthanasia) or found dead. Overall there was an increase in incidence in mortality in the Group 4 (control-dosed) and Group 7 (H4H14401P-dosed) animals as compared to the Group 2 controls and the other test protein dose groups. The gross observations and microscopic findings in all early death animals were similar in severity and character to those noted in the animals that survived to terminal euthanasia. Atrial dilatation/congestion was an additional finding not identified in animals that survived to scheduled terminal. This finding was considered to be associated with cardiovascular failure. The clinical signs observed generally did not occur following administration of test material and it is likely that most of the clinical signs were secondary to the development of hypertension, renal disease and cardiovascular complications/failure.

All of the test proteins caused a robust reduction in blood pressure on Day 21 which was followed by a compensatory increase in heart rate. On Day 22, only H4H14371P, H4H14357P, and H4H14401P caused a reduction in blood pressure and the magnitude of the change was reduced compared to the Day 21 response. On Day 35, only Comparator 1, H4H14357P, and H4H14371P caused blood pressure reductions at dose ˜4 times higher than the Day 21 doses.

Cardiovascular and renal parameters were altered as expected with the administration of the high salt diet. The high salt diet was associated with hypertension, increased urine output, and decreased renal function. All of the test proteins resulted in blood pressure reductions at 1 or more dose levels; however, H4H14357P, Comparator 1, and H4H14371P caused blood pressure reductions at more doses and generally with the greatest magnitude. H4H14357P appeared to have the best profile for reducing blood pressure at a range of doses. None of the test proteins altered renal function or protected from microscopic changes in the kidney associated with the model.

### Example 9: Pharmacokinetic Studies

The pharmacokinetic clearance rates of anti-MBG antigen-binding proteins were determined in C57BL/6 mice (Taconic Biosciences). Cohorts contained five mice per test protein and all mice received a single sub-cutaneous (1 mg/kg) dose. Blood samples were collected at 6 hours, 1, 2, 3, 4, 8, 11, 15, 21, 30, and 49 days post dosing.

Circulating drug levels were determined by total human antibody analysis using an ELISA immunoassay. Briefly, a goat anti-human IgG polyclonal antibody (Jackson ImmunoResearch Laboratory) was coated onto a 96-well Maxisorb plate (VWR) in order to capture the human IgG present in the sera. Plates were coated at 4° C. overnight, followed by non-specific blocking by BSA (Sigma). The serum samples containing test proteins were plated using a six-dose serial dilution and the reference standards of the dosed test proteins were plated in 12-dose serial dilution and incubated for one hour at room temperature. Following a washing step, the plate bound proteins were detected using a goat anti-human IgG polyclonal antibody conjugated with horseradish peroxidase (Jackson ImmunoResearch Laboratory) and incubated for one hour at room temperature followed by development with a colorimetric substrate such as BD OptEIA (BD Biosciences). After the reaction was stopped with 1M phosphoric acid, optical absorptions at 450 nm were recorded. Drug antibody concentrations in the sera were calculated based on the reference standard curves generated using GraphPad Prism software.

Pharmacokinetic parameters (elimination half-life, time of maximum concentration, maximum concentration and bioavailability) were calculated from the serum concentration-time data using non-compartmental analysis by Phoenix WinNonLin software (Pharsight).

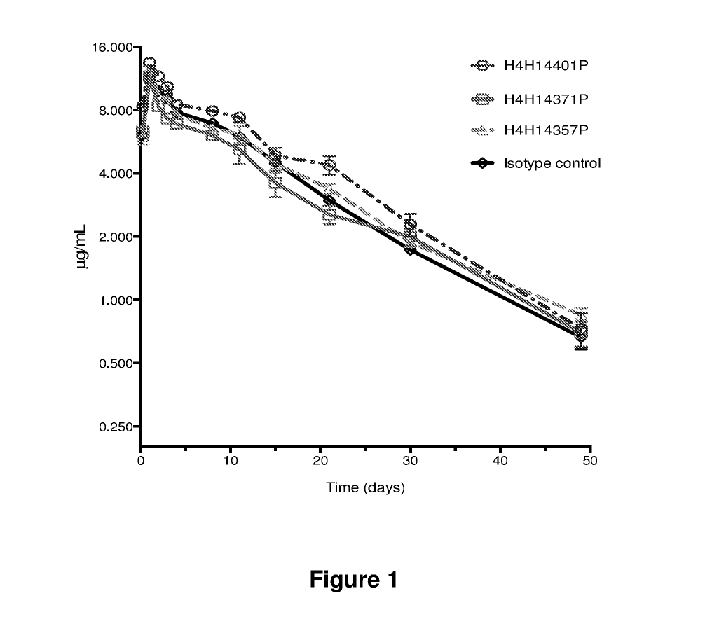

To examine the in vivo stability of three anti-MBG antigen-binding proteins, five C57BL/6 mice each were dosed with H4H14401P, H4H14371P, H4H14357P or isotype control. Each protein was dosed subcutaneously at 1 mg/kg and the time-course of serum concentration was determined (FIG. 1). Results showed that the three anti-MBG antigen-binding proteins have clearance profiles that are similar to the isotype control out to almost 50 days. The pharmacokinetic parameters were calculated from the serum concentration-time data (Table 8) and show that the bioavailability and half-life of the anti-MBG antigen-binding proteins are similar to those seen with the isotype control.

TABLE 8
Pharmacokinetic parameters

| Parameter | Units | H4H14401P | H4H14371P | H4H14357P | Control |
|---|---|---|---|---|---|
| Cmax | μg/mL | 14 ± 1.7 | 12 ± 1.3 | 12 ± 0.74 | 12 ± 0.57 |
| T½ | D | 12 ± 1.7 | 13 ± 0.87 | 13 ± 0.53 | 11 ± 1.8 |
| AUC | μg · h2/mL | 205 ± 26 | 137 ± 38 | 174 ± 19 | 160 ± 20 |
| tmax | d | 1.0 | 1.0 | 1.0 | 1.0 |

AUC: total area under the serum drug concentration-time curve;
Cmax: maximum serum drug concentration during a dosing interval;
T½: time required to divide the serum concentration by two after reaching equilibrium;
Tmax: time after drug administration when maximum serum concentration is reached

The maximum concentration (Cₘₐₓ) and the time to reach the Cₘₐₓ are also comparable for all of the test proteins. The results confirm that in vivo, the anti-MBG antigen-binding proteins exhibit similar stability to that of isotype control mAb.

### Example 10: Solubility and Viscosity Studies of Anti-MBG Antigen-Binding Proteins

Centrifugal based concentration was used to determine the solubility of 3 anti-MBG antigen-binding proteins as a screening tool to study feasibility of providing a high concentration drug product for subcutaneous or intramuscular administration. Anti-MBG antigen-binding proteins (50-52 mg/mL) in sample buffer (10 mM Histidine, pH 5.5) were added to Amicon ultra centrifugal filter tubes (Ultracel-30K) at room temperature. The filter tubes were centrifuged in a tabletop centrifuge at 7000 rcf for 30 min. Samples were then centrifuged for an additional 30 min at 7000 rcf or until no further reduction in volume was observed. Concentration of mAb in the supernatant was determined by measuring absorbance of the undiluted sample at 280 nm using a SoloVPE Slope spectrophotometer (C Technologies, Bridgewater, N.J., USA). Viscosity at high concentration of anti-MBG protein samples concentrated by ultracentrifugation were diluted to 175 mg/mL with buffer composed of 10 mM Histidine, pH 5.5 to measure viscosity. Viscosities were measured at 20° C. using a m-VROC viscometer (Rheosense Inc, San Ramon, Calif., USA). Reduction in viscosity by addition of viscosity reducing agent was also evaluated by compounding the antigen-binding proteins at 175 mg/mL protein with the viscosity reducing agent.

No precipitation was observed in the experiment. The highest concentration achieved for the anti-MBG protein molecules in this study was 333, 305, 287 and 185 mg/mL for H4H14357P, H4H14371P, H4H14401P and isotype control respectively. To further evaluate the potential feasibility of providing a high concentration drug product, the concentrated samples were diluted to 175 mg/mL for viscosity measurement. This concentration was chosen since it was achievable with all the antigen-binding proteins in this study. A viscosity of 20 cp or lower is desirable for subcutaneous drug products for ease of administration. Highest viscosity of 17.4 cp was observed for isotype control. The viscosity for H4H14357P, H4H14371P, and H4H14401P were 9.6, 8.8 and 14.1 respectively. Further, the impact of a viscosity reducing agent on the viscosity was investigated. Samples from ultracentrifugation were compounded with a viscosity reducing agent. As expected, the viscosity reducing agent significantly reduced the viscosity of isotype control from 17.4 cp to 11.2 cp and had modest impact on other mAb samples tested.

### Example 11: Agitation and Stability Studies

Anti-MBG antigen-binding proteins at 10 mg/mL in Type I glass vials were agitated on an orbital shaker (250 rpm, Chemglass Life Sciences, IS-500 Incubator Shaker) at room temperature for 24 to 48 hr with or without the presence of a non-ionic surfactant. Soluble aggregates were analyzed by size-exclusion ultra-high performance chromatography (SE-UPLC). Chromatograms were integrated and processed in Empower 3 software (Water Corp, Milford, Mass., USA). To determine degradation pathways and stability under accelerated conditions, 50 mg/mL solutions of Anti-MBG antigen-binding proteins were incubated at 45° C. for up to 28 days. Soluble aggregates were analyzed by SE-UPLC as described above.

Air-liquid and solid-liquid interface may result in destabilization of proteins. Interfacial interaction may occur during shipping and handling. A preliminary assessment of agitation induced instability was performed by shaking H4H14357P and isotype control molecules for up to 48 hours. Samples were assessed by visual appearance at the end of agitation and also by SE-UPLC. Results obtained indicated that both H4H14357P and isotype control were unstable upon agitation. H4H14357P samples were cloudy with visible precipitation. SE-UPLC analysis showed a significant increase in high molecular weight species (19.5%) after 48 hours of agitation. The percentage peak area of high molecular weight species was determined by adding peak areas of all the peaks eluting before the monomeric species, and calculating as percent of total peak area. Non-ionic surfactants have been widely used to protect proteins from agitation induced instability. H4H14357P and isotype control were compounded with polysorbate 80 (0.2% w/v) and the experiment was repeated. No meaningful changes in visual appearance or percent high molecular weight species determined by SE-UPLC were observed for both of the antigen-binding proteins tested. Based on the results of this experiment, an interfacial stabilizer may be needed to maintain the purity of the antigen-binding proteins tested. The starting monomeric purity of H4H14357P and isotype control, as determined by the SE-UPLC technique, was 98.8% and 98.9% respectively. After incubation at 45° C. for 28 days, percent peak area of the monomeric species decreased as a percent of total area of all the peaks and formation of high molecular weight species were observed, indicating formation of aggregates.

### Example 12: Crystallization and Structure Determination

The Fab fragment of H4H14401P was mixed with synthetic marinobufagenin in a 3:1 molar excess of MBG over Fab. Initial crystallization trials with the H4H14401P Fab:MBG complex were not successful, so an additional Fab (known to bind Ck, which is present on the light chain of the H4H14401P Fab) was added to the complex. A 1:1 complex of the two Fabs was purified by size-exclusion chromatography, and MBG was added to this complex in a 3:1 molar excess of MBG over H4H14401P Fab. Diffraction-quality crystals of the 2 Fab+MBG complex grew in conditions containing 0.8 M ammonium sulfate and 0.1 M sodium citrate pH 5. These crystals were frozen in liquid nitrogen and data to 3.6 A collected at beamline 5.0.2 of the Advanced Light Source (Berkeley, Calif.). The structure was determined by molecular replacement (McCoy et al 2007, J. Appl. Crystallogr. 40: 658-674) using Fab subdomains with high sequence identity from PDB codes 4YHY, 5DQD, 4LRN, 1 EEQ, and 4WCY. Once the two-Fab structure had been well refined, the difference electron density for MBG was very clear, allowing placement and refinement of MBG molecules. All refinement was carried out using REFMACS (Murshudov et al 2011, Acta Crystallogr. D Biol. Crystallogr. 67: 355-67).

The 3.6 Å crystal structure of MBG bound to the Fab fragment of H4H14401P was determined. The arrangement of the variable domains of this Fab is very similar to that seen in Bence-Jones proteins; for example, the Ca RMSD of this Fab's VR1+VR2 is 1.1 Å compared to the two Vk chains of the Bence-Jones protein Len (PDB code SLVE). In contrast, the constant portion of this Fab (Ck+CH1) superimposes on a conventional Fab's constant portion very well (Ca RMSD of 0.71 Å to the IgG4 Fab in PDB code 5F90). The Fab has a deep pocket at the center of the CDR surface, like most Bence-Jones proteins, and one molecule of MBG is bound in this pocket. The MBG is contacted by a set of tyrosines and tryptophans from CDR5, CDR6, CDR2 and CDR3; although this Fab has an unusually long CDR1 (encoded by the Vk4-1 gene), this CDR does not contact the MBG. H4H14401P is specific for MBG and does not bind the related cardiac glycosides ouabain or digoxin. The structure shows that 04 of MBG, the site of attachment for saccharide chains in ouabain and digoxin, is buried at the bottom of the binding pocket. There is no room in the pocket for additional sugar moieties, thus explaining the selectivity of this antibody. The anti-kappa antibody used to aid crystallization binds to the linker between Vk and Ck of H4H14401P, as well as to parts of Ck. The constant domain (Ck+CH1) of this anti-kappa antibody is not well ordered in this structure due to a lack of stabilizing crystal contacts, and its position should be considered approximate.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A recombinant antigen-binding protein comprising an antigen-binding domain that specifically binds to marinobufagenin (MBG) with a dissociation constant (K_{D}) of less than 50 nM as measured in an isothermal titration calorimetry assay at 25° C.

2. The antigen-binding protein of claim 1, wherein the antigen-binding protein shows one or more characteristics selected from the group consisting of:
(a) binds to MBG with a dissociation constant (K_{D}) of less than 25 nM, as measured in a isothermal titration calorimetry assay at 25° C.;
(b) binds to MBG with a dissociation constant (K_{D}) of less than 10 nM, as measured in a surface plasmon resonance assay at 25° C.;
(c) blocks binding of MBG to Na+/K+ ATPase;
(d) releases inhibition of Na+/K+ ATPase and facilitates membrane repolarization of a cell with EC₅₀ less than 300 nM, less than 200 nM, less than 150 nM or less than 100 nM, as measured in a membrane potential assay;
(e) does not bind to digitalis or digoxin;
(f) is fully human; and
(g) the antigen-binding domain comprises at least one immunoglobulin variable region comprising three complementarity determining regions (CDRs).

3. The antigen-binding protein of claim 2, wherein the at least one immunoglobulin variable region is not a heavy chain variable region.

4. The antigen-binding protein of any one of claims 1-3, wherein the antigen-binding domain comprises a first variable region (VR1) and a second variable region (VR2), wherein VR1 comprises three CDRs (CDR1, CDR2 and CDR3) and has an amino acid sequence selected from the group consisting of SEQ ID NOs: 2, 18, 34, 50, 66, 82, 98, 114, 130, 146, and 162; and VR2 comprises three CDRs (CDR4, CDR5 and CDR6) and has an amino acid sequence selected from the group consisting of SEQ ID NOs: 10, 26, 42, 58, 74, 90, 106, 122, 138, 154, and 170.

5. The antigen-binding protein of claim 4, comprising:
(a) a CDR1 domain having an amino acid sequence selected from the group consisting of SEQ ID NOs: 4, 20, 36, 52, 68, 84, 100, 116, 132, 148, and 164;
(b) a CDR2 domain having an amino acid sequence selected from the group consisting of SEQ ID NOs: 6, 22, 38, 54, 70, 86, 102, 118, 134, 150, and 166;
(c) a CDR3 domain having an amino acid sequence selected from the group consisting of SEQ ID NOs: 8, 24, 40, 56, 72, 88, 104, 120, 136, 152, and 168;
(d) a CDR4 domain having an amino acid sequence selected from the group consisting of SEQ ID NOs: 12, 28, 44, 60, 76, 92, 108, 124, 140, 156, and 172;
(e) a CDR5 domain having an amino acid sequence selected from the group consisting of SEQ ID NOs: 14, 30, 46, 62, 78, 94, 110, 126, 142, 158, and 174; and
(f) a CDR6 domain having an amino acid sequence selected from the group consisting of SEQ ID NOs: 16, 32, 48, 64, 80, 96, 112, 128, 144, 160, and 176.

6. The antigen-binding protein of claim 5, comprising a VR1/VR2 amino acid sequence pair selected from the group consisting of SEQ ID NOs: 2/10, 18/26, 34/42, 50/58, 66/74, 82/90, 98/106, 114/122, 130/138, 146/154, and 162/170.

7. The antigen-binding protein of any one of claims 4-6, wherein VR1 is a heavy chain variable region and VR2 is a light chain variable region.

8. The antigen-binding protein of any one of claims 4-6, wherein VR1 is a light chain variable region and VR2 is a light chain variable region.

9. The antigen-binding protein of any one of claims 1-8 further comprising a Fc domain.

10. The antigen-binding protein of claim 9, wherein the Fc domain is an IgG1 isotype.

11. The antigen-binding protein of claim 9, wherein the Fc domain is an IgG4 isotype.

12. An antigen-binding protein that competes for binding to MBG with an antigen-binding protein of claim 6.

13. The antigen-binding protein of any one of claims 1-12, wherein the antigen-binding protein is a multi-specific antigen-binding molecule.

14. A pharmaceutical composition comprising an antigen-binding protein of any one of claims 1-13 and a pharmaceutically acceptable carrier or diluent.

15. An isolated polynucleotide molecule comprising a polynucleotide sequence that encodes VR1 of an antigen-binding protein as set forth in any one of claims 4-13.

16. An isolated polynucleotide molecule comprising a polynucleotide sequence that encodes VR2 of an antigen-binding protein as set forth in any one of claims 4-13.

17. A vector comprising the polynucleotide sequence of claim 15 or 16.

18. A cell expressing the vector of claim 17.

19. A method of producing an antigen-binding protein of any one of claims 1-13 comprising culturing a cell of claim 18 under conditions permitting production of the antigen-binding protein and recovering the antigen-binding protein so produced.

20. A method of preventing, treating or ameliorating at least one symptom or indication of a MBG-associated disease or disorder, the method comprising administering a pharmaceutical composition comprising a therapeutically effective amount of an antigen-binding protein of any one of claims 1-13 to a subject in need thereof.

21. The method of claim 20, wherein the MBG-associated disease or disorder is selected from the group consisting of volume expansion hypertension, myocardial fibrosis, uremic cardiomyopathy, heart failure, myocardial infarction, renal failure, renal fibrosis and pre-eclampsia.

22. The method of claim 20 or 21, wherein the at least one symptom or indication is selected from the group consisting of high blood pressure, atherosclerosis, hypertension, angina, shortness of breath, palpitations in the chest, weakness or dizziness, nausea, sweating, pressure or pain in the chest, arm or below the breastbone, irregular heartbeat, and death.

23. The method of any one of claims 20-22, wherein the pharmaceutical composition is administered in combination with a second therapeutic agent; wherein the second therapeutic agent is selected from the group consisting of an anti-hypertensive drug, a statin, aspirin, a different antigen-binding protein to MBG, and a dietary supplement such as anti-oxidants.

24. The method of any one of claims 20-23, wherein the pharmaceutical composition is administered subcutaneously, intravenously, intradermally, intraperitoneally, orally, intramuscularly or intracranially.
